(19) United States
(12) Patent Application Publication
(10) Pub. No.: US 2007/0053831 A1
(43) Pub. Date: Mar. 8, 2007

(54) Methods for binding agents to b-amyloid plaques

(76) Inventors: Jorge R. Barrio, Agoura Hills, CA (US); Andre J. Petric, Ljubljana (SI); Nagichettiar Satyamurthy, Los Angeles, CA (US); Vladimir Kepe, Los Angeles, CA (US); Gary W. Small, Los Angeles, CA (US)

Correspondence Address: CHRISTIE, PARKER & HALE, LLP, PO BOX 7068, PASADENA, CA, 91109-7068, US

(21) Appl. No.: 10/557,655

(22) PCT Filed: May 20, 2004
(86) PCT No.: PCT/US04/16038

§ 371 Date: Nov. 21, 2005

Related U.S. Application Data
(60) Provisional application No. 60/471,945, filed on May 20, 2003.

(51) Int. Cl. A61K 51/00; C07D 475/02

(52) U.S. Cl. 424/1.11; 544/250; 544/255

(57) ABSTRACT

A method for labeling structures, such as β-amyloid plaques and neurofibrillary tangles, in vivo or in vitro, is provided and comprises contacting brain tissue with one or more compounds, preferably radiolabeled for detection by positron emission tomography (PET).

### CROSS REFERENCE TO RELATED APPLICATION

This application claims priority of U.S. Application No. 60/471,945, filed May 20, 2003, the entire content of which (including Appendices A and B) is incorporated by reference herein as if set forth in its entirety.

### ACKNOWLEDGMENT OF GOVERNMENT SUPPORT

This invention was made with government support under Grant No. DE-FC0387-ER60615, awarded by the United States Department of Energy. The government has certain rights in the invention.

### BACKGROUND OF THE INVENTION

Alzheimer's disease affects approximately 20 to 40% of the population over 80 years of age, the fastest growing age group in the United States and other post-industrial countries. Common features in the brain of patients with Alzheimer's disease include the presence of abundant intraneuronal neurofibrillary tangles (NFTs) and extracellular amyloid rich β-amyloid plaques. NFTs are cytoskeletal pathologies largely composed of aggregates of hyperphosphorylated tau proteins assembled into periodically restricted amyloid fibers called paired helical filaments. The major component of amyloid plaques is a peptide, a small 3943 aminoacid long β-amyloid peptide that is generated from the cleavage of a larger amyloid precursor protein. However, except for diffuse plaques formed almost exclusively of β-amyloid peptides, amyloid plaques are complex lesions containing numerous associated cellular products. Mutations causing increased production of the 42 amino acid form of this peptide have been genetically linked to autosomal dominant familial forms of Alzheimer's diseases. Deposits of β-amyloid occur very early in the disease process, long before clinical symptoms develop. Because these mutations appear to be pathogenic and cause Alzheimer's diseases in transgenic mice, β-amyloids are widely believed to play a causal role in the disease. Whether or not amyloid deposits are causal, they are certainly a key part of the diagnosis. Further, because amyloid plaques occur early in the disease, the ability to image deposits would provide a convenient marker for early diagnosis and prevention of the disease as well as a method for monitoring the effectiveness of therapeutic regimens.

Alzheimer's disease is currently definitively diagnosed by taking sections from postmortem brain and quantifying the density of neocortical amyloid deposits. Unfortunately, current techniques for detecting amyloid deposits and/or NFTs require postmortem or biopsy analysis. For example, thioflavin fluorescent-labeling of amyloid in brain sections in vitro is currently a widely-used method for evaluation of the brain. Another potential amyloid probe, Chrysamine-G, a congo red derivative, has also been developed. Congo red is a charged molecule and thus lacks sufficient hydrophobicity for diffusion through the blood brain barrier and is therefore not useful as an in vivo label. See Klunk et al, Neurobiology of Aging, 16:541-548 (1995), and PCT Publication No. WO 96/34853. Chrysamine G enters the blood brain barrier better than Congo red, but its ability to label amyloid plaques in Alzheimer's brain appears weak. See for example, H. Han, C-G Cho and P. T. Lansbury, Jr J. Am. Chem. Soc. 118, 4506 (1996); N. A. Dezutter et al, J. Label. Compd. Radiopharm. 42, 309 (1999). Similarly, earlier attempts to use monoclonal antibodies as probes for in-vivo imaging of β-amyloid were hampered by their limited ability to cross the blood brain barrier. See R. E. Majocha et al, J. Nucl. Med. 33, 2184 (1992). More recently, the use of monobiotinylated conjugates of 1251-Aβ 1-40 with permeability through the blood brain barrier has also been proposed (See Y. Saito et al., Proc. Natl. Acad. Sci. USA 22, 2288 (1991)), but its ability to label β-amyloid plaques and/or NFTs in vivo has not yet been demonstrated. Quantitation of the deposits in vivo is not yet possible with the currently available probes. Accordingly, a need exists for a convenient marker for early diagnosis of Alzheimer's disease.

In vivo, non invasive determination of regional cerebral glucose metabolic rates (rCMRG1) with positron emission tomography (PET) has been an important tool in the assessment of brain function in Alzheimer's disease patients. Numerous studies using 2-[F-18]fluoro-2-deoxy-D-glucose (FDG) have demonstrated a characteristic metabolic pattern of hypometabolism in temporoparietal and frontal association areas. A few of these studies have compared RCMRG1 with postmortem regional neuronal pathology. These results and the uncertainties of the Alzheimer's disease pathogenic cascade highlight the importance of assessing amyloid and neurofibril deposition in vivo, non-invasively in these patients.

### DESCRIPTION OF THE DRAWINGS

These and other features and advantages of the present invention will be better understood by reference to the following detailed description when considered in conjunction with the accompanying drawings wherein:

FIG. 1A shows 2-(1,1-dicyanopropen-2-yl)-6-dimethylaminonaphthalene (DDNP) fluorescence (ex 490 nm, em 520-530 nm) of amyloid plaques labeled in the cortex of the brain of an Alzheimer's disease patient (X400).

FIG. 1B shows strong DDNP labeling of plaques and weak DDNP labeling of tangles in the cortex of the brain of an Alzheimer's disease patient (X640).

FIG. 1C shows DDNP labeling of a single, large plaque with an amyloid core in human brain (X640).

FIG. 1D shows DDNP labeling of a plaque in agent Tg2576 HuAPPsw transgenic mouse brain (X500).

FIG. 1E shows Thioflavin S labeling of a cored plaque in Alzheimer's disease human brain (X640).

FIG. 1F shows 4G8 antibody labeling amyloid O-protein of a slice of the same human brain shown in FIG. 1E (X640).

FIG. 2A shows labeling of amyloid injected into rat brain, where an aliquot of β-amyloid 1-40 was allowed to aggregate for 8 days at 37° C., dried onto a gelatin coated slide, and labeled with DDNP, demonstrating fibrillar fluorescence consistent with amyloid.

FIG. 2B shows labeling of amyloid injected into rat brain, where 8 days after unilateral stereotaxic injection of 3 μg of aggregated β-amyloid 1-40 into rat cortex, the rats were injected with 100 μL of 640 μM DDNP into the carotid artery, anesthetized, and sacrificed by perfusion after 20 minutes and the brains were cryosectioned and examined for fluoroescence; FIG. 2B demonstrates in vivo DDNP fluorescently labeled amyloid at the tip of the need track (X100).

FIG. 2C shows a high power view of the in vivo DDNP labeled material of FIG. 2B (X200).

FIG. 2D depicts how formic acid treatment of a section through the injection site removes fluorescent labeling (X100).

FIG. 2E demonstrates that DDNP labeling is weak contralateral to the amyloid injection site, where no amyloid is present (X200).

FIG. 3A is a PET-[F-18]FDDNP (2-(1.1-dicyanopropen-2-yl)-6-(2-[¹⁸F]-fluoroethyl)-methylamino)-naphthalene) image of a brain cross-section through the hippocampus-amygdala-entorhinal/temporal cortex region of an Alzheimer's disease patient.

FIG. 3B is a PET-FDG (FDG is 2-[F-18]fluoro-2-deoxy-D-glucose) image of the brain cross-section of FIG. 3A.

FIG. 3C is an MRI image (proton relaxation times) of the brain cross-section of FIG. 3A.

FIG. 4 is a graph showing the estimated residence times of [F-18]FDDNP in pateints.

FIG. 5 shows an image (central image) obtained by immunostaining a forty five micrometer cryostate temporal cortex section of an Alzheimer's disease patient incubated with AT8 (anti-phosphotau) and 10G4 (anti-AB 1-15) at 1:800. Insets are adjacent sections of the same Alzheimer's disease brain specimen stained with FDDNP showing, beginning in the upper left corner and moving clockwise, (1) neuritic plaques, (2) diffuse plaque, (3) vascular amyloid, (4) lense plaques and tangles, and (5) dense tangles.

### DETAILED DESCRIPTION OF THE INVENTION

In one embodiment, the invention is directed to a method for labeling structures selected from the group consisting of β-amyloid plaques and neurofibrillary tangles either in vivo or in vitro, comprising contacting brain tissue with a compound of formula (IA) or (IB): 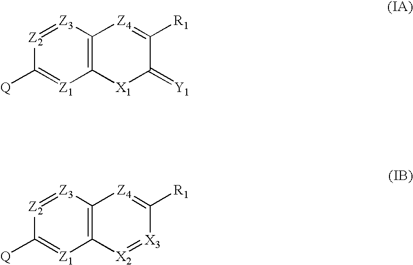
wherein:
Q is selected from the group consisting of —NR₂R₃, —OR₉ and —SR₉;
X₁ is selected from the group consisting of O, S, —NR₄, —CH₂, —CH-alkyl, and —C(alkyl)₂;
X₂ is selected from the group consisting of N and —CR₉;
X₃ is selected from the group consisting of N and —C—Y₂;
Y, is selected from the group consisting of O, S, —NR₉, and —C(R₉)₂;
Y₂ is selected from the group consisting of R₉, —N(R₉)₂, —OR₉ and —SR₉;
Z₁, Z₂, Z₃, and Z₄ are each independently selected from the group consisting of N and —CR₉;
R₁ is selected from the group consisting of —CN, —C(=A₁)-A₂, alkyl-A₂, alkenyl-A₂, alkynyl-A₂, 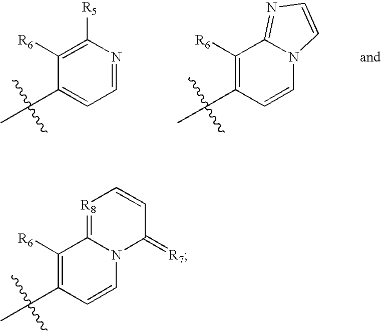
   wherein:
   A₁ is selected from the group consisting of O, S, —NR₄, —C(CN)₂, —C(CN)COOR₉, and —C(COOR₉)₂, and
   A₂ is selected from the group consisting of OH, —O-alkyl, —N₂, —NH—R₄, —N(R₄)₂, halogen, alkyl, cyclic ring, heterocyclic rings, —O-alkylenyl-R₄, —NH-alkenyl-R₄, -alkenyl-R₄, -alkenyl-NHR₄, -alkenyl-NH₂, -alkenyl-N(R₄)₂, —O-alkenyl-R₄, —NH-alkynyl-R₄, -alkynyl-R₄, -alkynyl-N₄, -alkynyl-NH₂, -alkynyl-N(R₄)₂,
or A₁ and A₂ together form a cyclic ring or heterocyclic ring,
or R₁ together with X₃ or Z₄ forms a cyclic ring or heterocyclic ring;
R₄ is a radical selected from the group consisting of hydrogen, alkyl, alkenyl, alkynyl, cyclic rings, heterocyclic rings, —C(O)O-alkyl, —C(O)O-alkenyl, —C(O)O-alkynyl, OH, OTs, and halogen;
R₅ is a radical selected from the group consisting of hydrogen, alkyl, alkenyl, alkynyl, cyclic rings, heterocyclic rings, —OH, —OTs, —SH, halogen, —N(R₄)₂, —O-alkyl, —O-alkenyl, —O-alkynyl, —S-alkyl, —S-alkenyl, and —S-alkynyl;
R₆ is a radical selected from the group consisting of hydrogen, —CN, —COOH, —C(O)O-alkyl, —C(O)O-alkylenyl-R₄, —C(O)-alkyl, —C(O)-alkylenyl-R₄, —C(O)-halogen, —C(O)NH₂, —C(O)NH-alkyl, —C(O)NH-alkylenyl-R₄;
R₇ is a radical selected from the group consisting of O, NH, and S;
R₈ is N; and
R₉ is a radical selected from the group consisting of hydrogen, alkyl, alkenyl, alkynyl, cyclic rings, heterocyclic rings; and
   R₂ and R₃ are each independently selected from the group consisting of hydrogen, alkyl, alkenyl, alkynyl, cyclic rings and heterocyclic rings,
or R₂ and R₃ together form a heterocyclic ring,
or R₃, together with Z₁ or Z₂ and the nitrogen to which R₃ is attached, forms a heterocyclic ring;
   wherein when the compound is of the formula (IB) and X₂, Z₁, Z₂, Z₃, and Z₄ are all CH, X₃ is not CH. In the above compounds, one or more of the hydrogen, halogen or carbon atoms may optionally be replaced with a radiolabel.

The invention is also directed to compositions that are useful in the above method and comprising a compound of formula (IA) or (IB):A method for labeling structures selected from the group consisting of β-amyloid plaques and neurofibrillary tangles either in vivo or in vitro, comprising contacting brain tissue with a compound of formula (IA) or (IB): 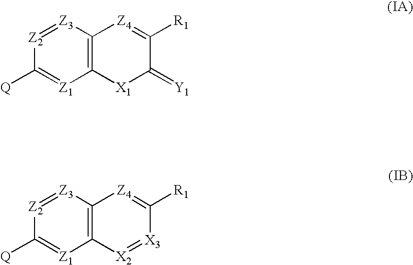
wherein:
Q is selected from the group consisting of —NR₂R₃, —OR₉ and —SR₉;
X₁ is selected from the group consisting of O, S, —NR₄, —CH₂, —CH-alkyl, and —C(alkyl)₂;
X₂ is selected from the group consisting of N and —CR₉;
X₃ is selected from the group consisting of N and —C—Y₂;
Y₁ is selected from the group consisting of O, S, —NR₉, and —C(R₉)₂;
Y₂ is selected from the group consisting of R₉, —N(R₉)₂, —OR₉ and —SR₉;
Z₁, Z₂, Z₃, and Z₄ are each independently selected from the group consisting of N and —CR₉;
R₁ is selected from the group consisting of —CN, —C(=A₁)-A₂, alkyl-A₂, alkenyl-A₂, alkynyl-A₂, 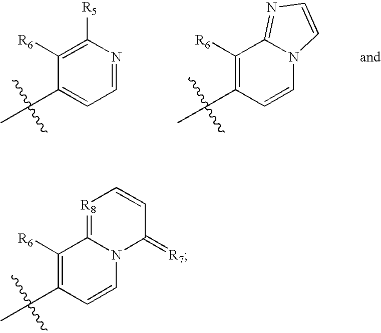
   wherein:
   A₁ is selected from the group consisting of O, S, —NR₄, —C(CN)₂, —C(CN)COOR₉, and —C(COOR₉)₂, and
A₂ is selected from the group consisting of OH, —O-alkyl, —NH₂, —NH—R₄, —N(R₄)₂, halogen, alkyl, cyclic ring, heterocyclic rings, —O-alkylenyl-R₄, —NH-alkenyl-R₄, -alkenyl-R₄, -alkenyl-NHR₄, -alkenyl-NH₂, -alkenyl-N(R₄)₂, —O-alkenyl-R₄, —NH-alkynyl-R₄, -alkynyl-R₄, -alkynyl-NHR₄, -alkynyl-NH₂, -alkynyl-N(R₄)₂,
   or A₁ and A₂ together form a cyclic ring or heterocyclic ring,
   or R₁ together with X₃ or Z₄ forms a cyclic ring or heterocyclic ring;
   R₄ is a radical selected from the group consisting of hydrogen, alkyl, alkenyl, alkynyl, cyclic rings, heterocyclic rings, —C(O)O-alkyl, —C(O)O-alkenyl, —C(O)O-alkynyl, OH, OTs, and halogen;
   R₅ is a radical selected from the group consisting of hydrogen, alkyl, alkenyl, alkynyl, cyclic rings, heterocyclic rings, —OH, —OTs, —SH, halogen, —N(R₄)₂, —O-alkyl, —O-alkenyl, —O-alkynyl, —S-alkyl, —S-alkenyl, and —S-alkynyl;
   R₆ is a radical selected from the group consisting of hydrogen, —CN, —COOH, —C(O)O-alkyl, —C(O)O-alkylenyl-R₄, —C(O)-alkyl, —C(O)-alkylenyl-R₄, —C(O)-halogen, —C(O)NH₂, —C(O)NH-alkyl, —C(O)NH-alkylenyl-R₄;
   R₇ is a radical selected from the group consisting of O, NH, and S;
   R₈ is N; and
   R₉ is a radical selected from the group consisting of hydrogen, alkyl, alkenyl, alkynyl, cyclic rings, heterocyclic rings; and
R₂ and R₃ are each independently selected from the group consisting of hydrogen, alkyl, alkenyl, alkynyl, cyclic rings and heterocyclic rings,
   or R₂ and R₃ together form a heterocyclic ring,
   or R₃, together with Z₁ or Z₂ and the nitrogen to which R₃ is attached, forms a heterocyclic ring;

wherein when the compound is of the formula (IB) and X₂, Z₁, Z₂, Z₃, and Z₄ are all CH, X₃ is not CH;

and further wherein one or more of the hydrogen atoms are optionally replaced with a radiolabel.

Definitions:

As used herein, the term “alkyl” refers to a straight or branched chain monovalent radical of saturated carbon atoms and hydrogen atoms, such as methyl, ethyl, propyl, isopropyl, butyl, isobutyl, t-butyl, pentyl, and hexyl, that may or may not be substituted. The term “lower alkyl” refers to a straight or branched chain monovalent radical having from one to four saturated carbon atoms and hydrogen atoms, such as methyl, ethyl, propyl, isopropyl, butyl, isobutyl, and t-butyl, that may or may not be substituted.

As used herein, the term “alkylenyl” refers to a straight or branched chain radical of carbon atoms containing at least one —(CH₂)— group that may or may not be substituted, such as ethylenyl and propylenyl.

As used herein, the term “alkenyl” refers to a straight or branched chain radical of carbon atoms containing at least one carbon-carbon double bond that may or may not be substituted, such as butenyl and pentenyl.

As used herein, the term “alkynyl” refers to a straight or branched chain radical of carbon atoms containing at least one carbon-carbon triple bond that may or may not be substituted, such as ethynyl, propynyl, butynyl, and pentynyl.

As used herein, the term “cyclic ring” refers to a saturated or unsaturated, monocyclic or polycyclic radical containing 3, 4, 5, 6, 7, 8, 9, 10, 11, or 12 ring atoms, each of which is saturated or unsaturated, that may or may not be substituted, such as cyclobutyl, cyclopentyl, cyclohexyl, cyclohexenyl, and phenyl.

As used herein, the term “heterocyclic ring” refers to a saturated or unsaturated, monocyclic or bicyclic radical containing 3, 4, 5, 6, 7, 8, 9, 10, 11, or 12 ring atoms, each of which is saturated or unsaturated, including 1, 2, 3, 4, or 5 heteroatoms selected from nitrogen, oxygen and sulfur, that may or may not be substituted. Nonlimiting examples include aziridine, azetidine, pyrrolidine, piperidine, and piperizine.

As noted above, the terms “alkyl,” “alkenyl,” “alkynyl,” “cyclic ring,” and “heterocyclic ring” include substituted alkyl, alkenyl, alkynyl, cyclic ring and heterocyclic ring groups. Such groups can be substituted by an suitable substituent, including, alkyl, alkenyl, alkynyl, cyclic ring, heterocyclic ring, halogen, —OH, —OTs, O-alkyl, O-alkenyl, O-alkynyl, O-cyclic ring, O-heterocyclic ring, acyl, thioacyl, sulfonyl, mercapto, alkylthio, amino, alkylamino, dialkylamino, and carbomoyl groups.

For the compounds of formula (I) and formula (II), preferably R₂ and R₃ are each independently alkyl, more preferably lower alkyl. For the compounds of formula (II), preferably R₉ is lower alkyl, more preferably methyl or ethyl, aryl and substituted aryl. Particularly preferred compounds for use in connection with the invention are 2-(1,1-dicyanopropen-2-yl)-6-dimethylaminonaphthalene (DDNP) and 2-(1,1-dicyanopropen-2-yl)-6-(ethyl)(methyl)(amino)-naphthalene, both of which can be optionally radiolabeled. Another preferred compound, particularly for use in vivo, is 2-(1.1-dicyanopropen-2-yl)-6-(2-[¹⁸F]-fluoroethyl)-methylamino)-naphthalene ([F-18]FDDNP).

The present invention is also directed to methods for detecting structures, such as β-amyloid plaques and neurofibrillary tangles in vitro and in vivo. The term “structures” refers to aggregates of biological materials containing peptides and other cellular materials that may occur as part of a disease pathology. The term “peptides” includes proteins.

The compounds described above have fluorescent activity in the range of about 470 to 610 nm. In one application, the present invention labels β-amyloid plaques and neurofibrillary tangles in brain tissue. Accordingly, for in vitro detection Alzheimer's disease, the compounds are contacted with brain tissue, and the brain tissue observed with a fluorescence microscope.

For in vivo detection, preferably the compounds are radiolabeled. A preferred radio label is ¹⁸F, which has a half-life of approximately two hours for position emission tomography (PET). Another radio label is radioiodine, for example, ¹²³I for use with single photon emission computed tomography (SPECT). Alternatively, other radiolabels are used, such as ¹¹C, ¹³N and ¹⁵O, although these radiolabels are less desirable due to their relatively short half-lives. Any atom in the compound can be replaced with a suitable radiolabel. Radiolabeling can be achieved by any method known to those skilled in the art. For example, dry [F-18]fluoride ion [¹⁸O(p,n)¹⁸F] in K₂CO₃ (0.75 mg) and Kryptofix 2.2.2™ (19 mg) are added to a solution of the compound of formula (I) or formula (II) (4 mg in 1 mL CH₃CN). The mixture is heated in an oil bath at 85° C. for about 10 to 40 minutes. After cooling and dilution with water, the radiolabeled product can be purified by preparative HPLC. Kryptofix 2.2.2™ is a crown ether, available from Aldrich Chemical Co. (Milwaukee, Wis.).

A solution containing the radiolabeled compound is then injected into the patient. As used herein, the term “patient” refers to any mammal, including humans, rats, mice, dogs and cats. Neuroanatomical regions can be determined manually using MRI scans, for example, using a Tela magnet, and then on amyloid-PET (positron emission tomography) and FDG-PET (fluorodeoxyglucose-PET) by coregistration of the MRI scans. PET has current resolution of 2 to 3 min, a dynamic determination of radiolabeled compound deposition in the brain, and permits detection of abnormal areas.

By the above-described methods, diseases characterized by the accumulation of β-amyloid plaques and neurofibrillary tangles such as Alzheimer's disease and other diseases associated with brain deterioration, can be detected.

The invention is also directed to a method for determining the ability of a therapeutic agent to treat or prevent Alzheimer's disease in a patient. The phrase “prevent Alzheimer's disease” includes reducing the risk and/or delaying the onset of Alzheimer's disease. The method involves contacting, in vivo or in vitro, β-amyloid peptide with the therapeutic agent and a radiolabeled compound according to formula (IA) or (IB): 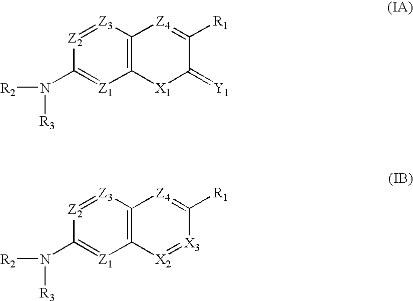
wherein:
X₁ is selected from the group consisting of O, S, —NR₄, —CH₂, —CH-alkyl, and —C(alkyl)₂;
X₂ is selected from the group consisting of N, —CH, and —C-alkyl;
X₃ is selected from the group consisting of O, S, —NR₄, and —C—Y₂;
Y₁ is selected from the group consisting of O, S, —NH, —CH₂, —CH-alkyl, and —C(alkyl)₂;
Y₂ is selected from the group consisting of H, OH, SH, —NH₂, —NH-alkyl, —N(alkyl)₂, —O-alkyl, and alkyl;
Z₁, Z₂ Z₃, and Z₄ are each independently selected from the group consisting of N, —CH, and —C-alkyl;
R₁ is selected from the group consisting of —CN, —C(=A₁)-A₂, 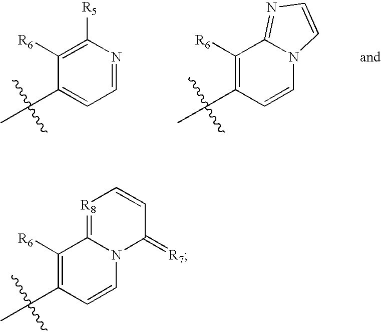
   wherein:
   A₁ is selected from the group consisting of O, S, —NR₄, —C(CN)₂, —C(CN)COOR₄, and —C(COOR₄)₂, and
   A₂ is selected from the group consisting of OH, —O-alkyl, —NH₂, —NH—R₄, —N(R₄)₂, halogen, alkyl, aryl, heterocyclic rings, —O-alkylenyl-R₄, —NH-alkylenyl-R₄, -alkylenyl-R₄, -alkylenyl-NHR₄, -alkylenyl-NH₂, and -alkylenyl-N(R₄)₂,
   or A₁ and A₂ together form an aryl or heterocyclic ring,
   or R₁ together with X₃ or Z₄ forms an aryl or heterocyclic ring substituted with an A₁ substituted at a carbon other than R₁, X₃ or Z₄;
   R₄ is a radical selected from the group consisting of alkyl, substituted alkyl, aryl, substituted aryl, —C(O)O-alkyl, OH, OTs, and halogen;
   R₅ is a radical selected from the group consisting of —NH₂, —OH, —OTs, —SH, halogen, —NH-alkyl, —NHR₄, —NH-alkylenyl-R₄, alkyl-(O-alkyl)₂, —O-alkyl, —O-alkylenyl-R₄, —S-alkyl, and —S-alkylenyl-R₄;
   R₆ is a radical selected from the group consisting of —CN, —COOH, —C(O)O-alkyl, —C(O)O-alkylenyl-R₄, —C(O)-alkyl, —C(O)-alkylenyl-R₄, —C(O)-halogen, —C(O)NH₂, —C(O)NH-alkyl, —C(O)NH-alkylenyl-R₄;
   R₇ is a radical selected from the group consisting of O, NH, and S; and
   R₈ is N, and
R₂ and R₃ are each independently selected from the group consisting of alkyl and alkylenyl-R₁₀, wherein R₁₀ is selected from the group consisting of —OH, —OTs, halogen, spiperone, spiperone ketal, and spiperone-3-yl,
   or R₂ and R₃ together form a heterocyclic ring, optionally substituted with at least one radical selected from the group consisting of alkyl, alkoxy, OH, OTs, halogen, alkylenyl-R₅, carbonyl, spiperone, spiperone ketal and spiperone-3-yl,
   or R₃, together with Z₁ or Z₂ and the nitrogen to which R₃ is attached, forms a heterocyclic ring or a substituted heterocyclic ring;

wherein one or more of the hydrogen, halogen or carbon atoms is replaced with a radiolabel.

In accordance with the method, at least some of the radiolabeled compound binds to the β-amyloid peptide. In connection with this method, the terminology β-amyloid peptide includes β-amyloid aggregates or fibrils as well as β-amyloid senile plaques. The therapeutic agent and the radiolabeled compound can both be contacted with the β-amyloid peptide simultaneously, or the therapeutic agent can be contacted with the β-amyloid peptide before and/or after the radiolabeled compound is contacted with the β-amyloid peptide.

The amount of radiolabeled compound that did not bind to the β-amyloid peptide is then determined to thereby determine whether and to what extent the non-radioactive agent did bind to the β-amyloid peptide. In a preferred embodiment, a control value is obtained by contacting only the radiolabeled compound (i.e., not in the presence of the non-radioactive agent) with the β-amyloid peptide to determine 100% specific binding.

With this method, the concentration of the therapeutic agent can be varied to determine the extent to which the therapeutic agent may bind to the β-amyloid peptide.

In another method in accordance with the invention, the anti-aggregation effect of a therapeutic agent can be evaluated. The anti-aggregation effect refers to the ability of the therapeutic agent to destroy and/or prevent formation of β-amyloid peptide. With this method, the amount of β-amyloid peptide in a patient can be determined using a radiolabeled compound, as generally described above. Thereafter, a therapeutic agent can be administered to the patient over a period of time, such as a week, a month or longer, so that the therapeutic agent comes into contact with the β-amyloid peptide. After the period of time, the radiolabeled compound can again be contacted with the β-amyloid peptide to determine the amount of β-amyloid peptide present in the patient. For determining the extent to which the therapeutic agent prevents formation of β-amyloid peptide, it is useful to evaluate one or more patients who may be predisposed to Alzheimer's disease, but in whom in the disease has not significantly progressed.

The invention is also directed to a method for treating or preventing Alzheimer's disease in a patient. The method comprises administering to the patient a therapeutically effective amount of an agent according to formula (A) or (IB), described above.

### EXAMPLES

### Example 1

The following compositions according to the invention were prepared. NMR spectra were obtained on Bruker AM 360 WB or DPX 300 Spectrometers. ¹H chemical shifts are reported in ppm downfield from TMS as an internal standard. ¹⁹F chemical shifts are reported relative to external fluorotrichloromethane. Deuteriochloroform was used as the solvent unless stated otherwise. Melting points were determined on an Electrothermal Melting Point Apparatus and are uncorrected. Elemental analyses were performed by Galbraith Laboratories, Inc., Knoxville, Tenn. or Ms. Metka Kastelic at the Faculty of Chemistry and Chemical Technology, University of Ljubljana. Radial chromatography was performed on Chromatotron (Harrison Research, 840 Moana Court, Palo Alto, Calif. 94306). The rotors were prepared as recommended by Harrison Research using E. Merck Silica Gel (Cat. No. 7749-3). HPLC was performed on an Alltech Econosil C-18 5 μm, 4.6×250 mm column using a 40:60:2 mix of water: acetonitrile: triethyl amine as the solvent. UV detection at 254 nm was used. Solvents and reagents were from Fisher, Aldrich or Fluka and were used as received unless noted otherwise.

### Example 1(a)

### Preparation of 2-(1,1-dicyanopropen-2-yl)-6-dimethylaminonaphthalene (DDNP)

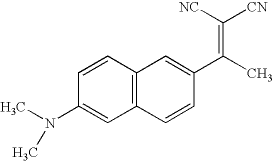

To a solution of 5.26 g (117 mmol) of dimethylamine in 29 mL of freshly distilled hexamethylphosphoric triamide (HMPT) were added 31 mL of dry toluene and 780 mg (112 mmol) of Li in small pieces. The mixture was stirred under argon at room temperature for 1.5 hours. 2-Acetyl-6-methoxynaphthalene was prepared as described in Arsenijevic et al., Org. Synth. Coll. 1988, 6:34-36, the disclosure of which is incorporated herein by reference. 2-Acetyl-6-methoxynaphthalene (5.57 g, 27.8 mmol) was added in one portion, and stirring was continued for 20 hours. The mixture was cooled in an ice-water bath and poured into a cold water/ethyl acetate mixture (300 mL each). After thorough mixing, the layers were separated, and the water layer was extracted twice with 225 mL of ethyl acetate. Organic extracts were combined, dried, and evaporated to give a yellow solid. Recrystallization from ethanol afforded 3.67 g (64%) of 2-acetyl-6-(dimethylamino)naphthalene (ADMAN) as a yellow solid, melting at 153.5-155° C.: ¹H NMR (CDCl₃, TMS) δ 2.67 (s, 3H, COCH₃), 3.15 (s, 6H, N(CH₃)₂), 6.87 (d, 1H, H-5), 7.17 (dd, 1H, H-7), 7.63 (d, 1H, H-4), 7.80 (d, 1H, H-8), 7.92 (dd, 1H, H-3), 8.32 (bs, 1H, H-1). J_{1,3}=2.3 Hz, J_{3,4}=8.7 Hz, J_{5,7}=2.4 Hz, J_{7,8}=9.3 Hz. MS (M⁺) 213: found: 213. Anal. Calcd for C₁₄H₁₅NO: C, 78.84: H, 7.09; N, 6.57. Found C, 78.96; H, 7.10; N, 6.45.

A mixture of malonitrile (436 mg, 6.6 mmol) and ADMAN (1.278 g, 6.6 mmol) was heated to 110° C. in 20 mL of pyridine for 19 hours. After cooling, the remaining red solid was dissolved in 100 mL of methylene chloride, adsorbed onto 10 g of flash silica get (230-400 mesh) and chromatographed with toluene. Appropriate fractions were combined and evaporated to give 1.12 g (72%) of 2-(1,1-dicyanopropen-2-yl)-6-dimethylaminonaphthalene (DDNP). Recrystallization from benzene-hexane gave red needles melting at 154.5-155° C.: ¹H NMR (CDCl₃, TMS) δ 2.69 (s, 3H, CH₃), 3.11 (s, 6H, N(CH₃)), 6.85 (d, 1H, H-5), 7.18 (dd, 1H, H-7), 7.56 (dd, 1H, H-3), 7.66 (d, 1H, H-4), 7.76 (d, 1H, H-8), 8.02 (d, 1H, H-1). J_{1,3}=2.04 Hz, J_{3,4}=9.13 Hz, J_{5,7}=2.5 Hz, J_{7,8}=9.11 Hz. IR (CHCl₃) 2250 cm⁻¹ (CN stretching). MS (M⁺) 261: found: 262. Anal. Calcd for C₁₇H₁₅N₃: C, 78.13: H, 5.79; N, 18.08. Found C, 78.17; H, 5.68; N, 17.91.

### Example 1(b)

### Preparation of 2-(1-{6[ethyl-(2-{8-[4-(4-fluorophenyl)-4-oxobutyl]-4-oxo-1-phenyl-1,3,8-triazaspiro[4.5]dec-3-yl}ethyl)-amino]-2-naphthyl}ethylidene)malononitrile

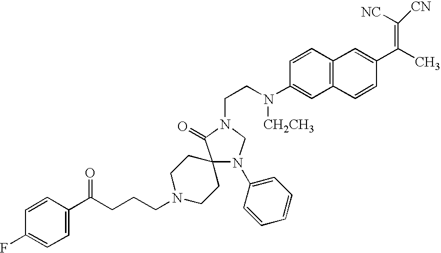

In a 3 L two-neck round bottom flask, equipped with a reflux condenser and a dropping funnel, 2 L of hydrochloric acid (d=1.16) were stirred and heated to boiling. A solution of 6.06 g (30.3 mmol) of 1-(6-methoxy-2-naphthyl)-1-ethanone (prepared as described in Arsenijevic et al., Org. Synth. Coll. 6:34 (1988), the disclosure of which is incorporated herein by reference) in a minimum amount of dichloromethane was added, and the mixture was stirred and heated at reflux for 2 hours. The hot solution was filtered through a mineral wool plug to remove oily residue. The solid that separated after cooling was filtered on a glass frit and dissolved in 130 mL of ethyl acetate. The solution was washed with brine, dried with anhydrous magnesium sulfate and evaporated to give 5 g (89%) of 1-(6-hydroxy-2-naphthyl)1-ethanone.

A mixture of 1-(6-hydroxy-2-naphthyl)ethanone (744 mg, 3.92 mmol), sodium hydrogen sulfated) (1.66 g, 16 mmol), 2-ethylaminoethanol (2 mL) and water (5 mL) was heated in a steel bomb at 130-140° C. for 3 days. After cooling, the mixture was distributed between water and ethyl acetate, and the organic layer was washed with brine, dried and evaporated. The residue was dissolved in acetone and loaded onto a 4 mm dry silica plate for radial chromatography. The plate was eluted with a 1:1 mixture of petroleum ether and ethyl acetate. Appropriate fractions were collected and evaporated to give 125 mg (12%) of 1-{6-[ethyl-(2-hydroxylethyl)-amino]-2-naphthyl}ethanone.

A solution of 1-{6-[ethyl-(2-hydroxylethyl)-amino]-2-naphthyl}ethanone (125 mg, 0.486 mmol) in pyridine (3.5 mL) was cooled to −15° C. and p-toluenesulfonic anhydride (252 mg, 0.81 mmol) was added with stirring under argon. The reaction mixture was allowed to slowly warm up to the room temperature, and stirring was continued for 24 hours. Because TLC (silica, 10% ethyl acetate in petroleum ether) revealed that the starting material was still present, more p-toluenesulphonic anhydride (252 mg, 0.81 mmol) was added, and stirring was continued for an additional 24 hours. The mixture was then cooled in an ice-water bath and distributed between brine and ether. The organic layer was dried and evaporated to leave an oily residue. The product, 6-acetyl-2-(ethyl-2-[(4-methylphenyl)-sulfonyloxy]-ethylamino)-naphthalene, was isolated by radial chromatography (1 mm silica, dichloromethane) in 30% yield. HRMS calcd. for C₂₃H₂₅NO₄S:411.1504. Found: 411.1514. ¹HNMR δ1.25 (t, 3H, CH₂CH₃), 2.33 (s, 3H, Ph-CH₃), 2.67 (s, 3H, COCH₃), 3.49 (q, 2H, CH₂CH₃), 3.75 (t, 2H, NCH₂CH₂O), 4.25 (t, 2H, NCH₂CH₂O), 6.97 (d, 1H, 5-H), 7.01 (dd, 1H, 7-H), 7.18 and 7.20 (d, 2H, 3′-H, 5′-H), 7.56 (d, 1H, 4-H), 7.69 and 7.72 (d, 2H, 2′-H, 6′-H), 7.75 (d, 1H, 8-H), 7.93 (dd, 1H, 3-H), 8.29 (d, 1H, 1-H). J_{1,3}=1.6 Hz, J_{2′,6}=J_{3′,5}=8.5 Hz, J_{7,5}=2.5 Hz, J_{7,8}=9.2 Hz, J_{3,4}=8.7 Hz, J_{(CH2CH3)}=7.1 Hz, J_{(NCH2CH2O)}=6.2 Hz.

To a solution of sodium hydroxide (1 g) and tetra-n-butylammonium hydrogensulfate (VI) (50 mg, 0.15 mmol) in water (2 mL), spiperone ketal (8-3[2-(4-fluorophenyl)-1,3-dioxolan-2-yl]propyl-1-phenyl-1,3,8-triazaspiro[4.5]decan-4-one (which can be prepared as described in U.S. Pat. No. 3,839,342, Chem Abstr. 82:43416, and Kiesewetter et al., Appl. Radiat. Isot. 37:1181 (1986), the disclosures of which are incorporated herein by reference) (15 mg, 0.034 mmol) was added and vigorously stirred. After 10 minutes, a solution of 6-acetyl-2-(ethyl-2-[(4 methylphenyl)-sulfonyloxy-ethylamino)-naphthalene (12 mg, 0.03 mmol) in toluene (3 mL) was added, and the reaction mixture was stirred and heated at 90° C. for 1 hour. After cooling, the reaction mixture was distributed between water and dichloromethane, and the organic layer was washed with brine, dried and evaporated to leave an oily residue. Radial chromatography (1 mm silica, 2% methanol in dichlormethane) yielded 5 mg (25%) of 1-[6-(ethyl-2-[(8-3-[2-(4-fluorophenyl)-1,3-dioxolan-2-yl]-propyl-4-phenyl-2,4,8-triazaspiro[4.5]dec-1-en-1-yl)-oxy]-ethylamino)-2-naphthyl]1-ethanone (compound A) and 11 mg (56%) of 1-[6-(ethyl-[2-(8-3-[2-(4-fluorophenyl)-1,3-dioxolan-2-yl]-propyl-1-oxo-4-phenyl-2,4,8-triazaspiro[4.5]dec-2-yl)-ethyl]amino-2-naphthyl)-1-ethanone (compound B).

Compound A—HRMS calcd. for C₄₁H₄₇FN₄O₄: 678.3581. Found: 678.3605. ¹H NMR: δ 1.45-2.24 (m, 11H, spiperone CH₂, CH₂CH₃), 2.35-2.84 (m, 6H, spiperone), 2.65 (s, 3H, OCH₃), 3.59 (q, 2H, NCH₂CH₃), 2.35-2.84 (M, 6H, spiperone), 2.65 (s, 3H, OCH₃), 3.59 (q, 2H, NCH₂CH₃), 3.76 in 4.05 (m, 4H, OCH₂CH₂O), 3.85 (t, 2H, NCH₂CH₂O), 4.52 (t, 2H, OCH₂CH₂N), 4.99 (s, 2H, NCH₂N), 6.76-6.83 9m, 3H, phenyl, fluorophenyl), 6.93 (d, 1H, 5-H), 6.95-7.04 (M, 2H, phenyl, fluorophenyl), 7.19 (dd, 1H, 7-H), 7.21-7.26 (m, 2H, phenyl, fluorophenyl), 7.39-7.45 (m, 2H, phenyl, fluoropheynl), 7.61 (d, 1H, 4-H), 7.78 (d, 1H, 8-H), 7.93 (dd, 1H, 3-H), 8.30 (d, 1H, 1-H). J_{1,3}=1.5 Hz, J_{5,7}=2.4 Hz, J_{3,4}=9.5 Hz, J=9.2 Hz, J_{(CH2CH3)}=7.1 Hz, J_{(NCH2CH2O)}=6.3 Hz.

Compound B—HRMS calcd. for C₄₁H₄₇FN₄O₄: 678.3581. Found: 678.3603. ¹H NMR: δ 1.20-1.94 (m, 17H, spiperone CH₂, CH₂CH₃), 2.66 (s, 3H, COCH₃), 3.56 (q, 2H, NCH₂CH₃), 3.66 and 4.02 (m, 4H, OCH₂CH₂O), 3.71-3.81 (m, 4H, NCH₂CH₂N), 4.68 (s, 2H, NCH₂N), 6.82-6.90 (m, 2H, phenyl, fluorophenyl), 6.94 (d, 1H, 5-H), 6.98-7.04 (m, 2H, phenyl, fluorophenyl), 7.18 (dd, 1H, H-7), 7.21-7.26 (m, 3H, phenyl, fluorophenyl), 7.39-7.45 (m, 2H, phenyl, fluorophenyl), 7.60 (d, 1H, 4-H), 7.78 (d, 1H, 8-H), 7.93 (dd, 1H, 3-H), 8.29 (d, 1H, 1-H). J_{1,3}=1.6 Hz, J_{3,4}=9.8 Hz, J_{5,7}=2.4 Hz, J_{7,8}=10.4 Hz, J_{(CH2CH3)}=7.1 Hz.

A solution of 1-[6-(ethyl-[2-(8-3-[2-(4-fluorophenyl)-1,3-dioxolan-2-yl]-propyl-1-oxo-4-phenyl-2,4,8-tri-azaspiro[4.5]dec-2-yl)-ethyl]amino-2-naphthyl)-1-ethanone (13 mg, 0.018 mmol) and malononitrile (6 mg, 0.09 mmol) in pyridine (3 mL) was heated at 85° C. under argon for 24 hours. After pyridine was removed in vacuo at room temperature, the residue was distributed between brine and dichloromethane, and the organic layer was dried and evaporated. The product, 2-[1-(6-ethyl-[2-(8-3-[2-(4-fluorophenyl)-1,3-dioxolan-2-yl]-propyl-1-oxo-4-phenyl-2,4,8-triazoaspiro[4,5]dec-2-yl)-ethyl]-amino-2-naphthyl)-ethylidene]-malononitrile was isolated by radial chromatography (1 mm silica, 2.5% methanol in dichloromethane; 13.5 mg, 97%).

HRMS calcd. for C₄₄H₄₈FN₆O₃ (M+H): 727.37719. Found: 727.3772. ¹H NMR: δ 1.25-1.93 (m, 17H, spiperone CH₂, CH₂CH₃), 2.70 (s, 3H, C═C—CH₃), 3.57 (q, 2H, NCH₂CH₃), 3.64 and 4.03 (m, 4H, OCH₂CH₂O), 3.71-3.78 (m, 4H, NCH₁CH₂N), 4.68 (s, 2H, NCH₂N), 6.83-6.88 (m, 2H, phenyl, fluorophenyl), 6.94 (d, 1H, 5-H), 6.96-7.04 (m, 2H, phenyl, fluorophenyl), 7.18 (dd, 1H, H-7), 7.21-7.25 (m, 3H, phenyl, fluorophenyl), 7.39-7.45 (m, 2H, phenyl, fluorophenyl), 7.56 (dd, 1H, 3-H), 7.63 (d, 1H, 4-H), 7.76 (dd, 1H, 9-H), 8.00 (d, 1H, 1-H). J_{1,3}=1.9 Hz, J_{3,4}=8.8 Hz, J_{5,7}=2.4 Hz, J_{7,8}=9.3 Hz, J_{(CH2CH3)}=7.1 Hz.

The ketal protective group was removed by stirring 2-[1-(6-ethyl-[2-(8-3-[2-(4-fluorophenyl)-1,3-dioxolan-2-yl]-propyl-1-oxo-4-phenyl-2,4,8-triazaspiro[4.53]dec-2-yl)-ethyl]-amino-2-naphthyl)-ethylidene]-malononitrile (13.5 mg, 0.0186 mmol) in methanol (1 mL) with one drop of concentrated hydrochloric acid for 3 hours at room temperature. The reaction mixture was diluted with dichloromethane and washed with a saturated solution of sodium bicarbonate. After evaporation in vacuo, the residue was purified by radial chromatography (1 mm silica, 2% methanol in dichloromethane) to give 10 mg (79%) of 2-(1-6[ethyl-(2-8-[4-(4-fluorophenyl)-4-oxobutyl)-1-oxo-4-phenyl-2,4,8-tiazaspiro[4.5]dec-2-ylethyl)-amino]-2-naphthylethylidene)-malonitrile. FAB MS calcd for C₄₂H₄₄FN₆O₂ (M+H): 683.35. Found 683. ¹H NMR: δ 1.21-3.02 (m, 17H, spiperone CH₂, CH₃), 2.71 (s, 3H, C═C—CH₃), 3.56 (q, 2H, NCH₃), 3.69 (m, 4H, NCH₂CH₂N), 4,67 (s, 2H, NCH₂N), 6.79-7.23 (m, 7H, phenyl, fluorophenyl), 6.95 (d, 1H, 5-H), 5,19 (dd, 1H, 7-H), 7.56 (dd, 1H, 3-H), 7.65 (d, 1H, 4-H), 7.76 (d, 1H, 8-H), 7.97-8.04 (m, 3H, fluorophenyl, 1-H). J_{1,3}=1.9 Hz, J_{3,4}=8.8 Hz, J_{5,7}=2.5 Hz, J_{7,8}=9.1 Hz, J_{(CH2CH3)}=7.1 Hz.

### Example 1(c)

### 2-(1-6-[4-(8-[4-(4-Fluorophenyl)-4-oxobutyl]-1-oxo-4-phenyl-2,4,8-triazaspiro[4.5]-dec-2-ylmethyl)=piperidino]-2-naphthylethylidene)-malononitrile

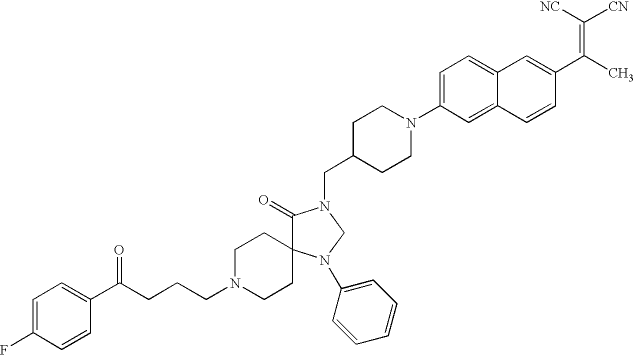

A mixture of 1-(6-hydroxy-2-naphthyl)-1-ethanone (653 mg, 3.5 mmol) (prepared as described in Example 1(b)), sodium hydrogensulfate(IV) (1.6 g, 15.5 mmol), 4-piperidylmethanol (2 g, 17.6 mmol) (prepared as described in Bradbury et al., J. Med. Chem. 34:1073 (1991), the disclosure of which is incorporated herein by reference), and water (6 mL) was heated in a steel bomb at 135-142° C. for 16 days. After cooling, the reaction mixture was extracted with ethyl acetate. Some product still remained in the residue, so it was further extracted with 5% methanol in dichloromethane. Organic extracts were combined, dried and evaporated. The residue was chromatographed by radial chromatography (2 mm silica, 2% methanol in dichloromethane) to yield 139 mg (14%) of 1-6-[(4-hydroxymethyl)-piperidino]-2-naphthyl-1-ethanone. After recrystallization from ethyl acetate the compound melted at 180-182° C. ¹H NMR: δ 1.44 (dddd, 2H, 3′ a-H, 5′a-H), 1.76 (m, 1H, 4′a-H), 1.91 (bd, 2H, 3′e-H, 5′c-H), 2.68 (s, 3H, COCH₃), 2.89 (ddd, 2H, 2′a-H, 6′a-H), 3.58 (d, 2H, OCH₂), 3.94 (bd, 2H, 2′e-H, 6′e-H), 7.10 (d, 1H, 5-H), 7.32 (dd, 1H, 7-H), 7.66 (d, 1H, 4-H), 7.80 (d, 1H, 8-H), 7.94 (dd, 1H, 3-H), 8.32 (d, 1H, 1-H) J_{3′a,3′e}=J_{5′a,5′e}12.5 Hz, J_{2′a,3′a}=J_{6′a,5′a}=12.5 Hz, J_{3′a,4′a}=J_{5′a,4′a}=12.5 Hz, J_{2′e,3′a}=J_{6′e,5′a}=4.0 Hz, J_{2a′,2′e}=J_{6′a,6′e}=12.5 Hz, J_{2′a,3′a}J=_{6′a,5′e}=2.6 Hz, J_{4′a,OCH2}=6.4 Hz, J_{1,3}=1.9 Hz, J_{3,4}=8.9 Hz, J_{5,7}=2.3 Hz, J_{7,8}=9.0 Hz.

A solution of 1-6-[(4-hydroxymethyl)-piperidino]-2-naphthyl-1-ethanone (59 mg, 0.2 mmol) in pyridine (3 mL) was cooled to −15° C., and p-toluenesulfonic anhydride (205 mg, 0.6 mmol) was added with stirring under argon. The reaction mixture was allowed to slowly warm up to the room temperature during 1 hour. It was cooled again and distributed between brine and ether. The organic layer was washed with brine, dried and evaporated to leave 83 mg (91%) of raw 1-(6-acetyl-2-naphthyl)-4-[(4-methylphenyl)-sulfonyloxy]-methylpiperidine.

To a solution of sodium hydroxide (1 g) and tetra-n-butylammonium hydrogen-sulfate(VI) (50 mg, 0.15 mmol) in water (2 mL), spiperone ketal (100 mg, 0.2 mmol) was added and vigorously stirred. After 10 minutes, a solution of 1-(6-acetyl-2-naphthyl)-4-[(4-methylphenyl)-sulfonyloxy]-methylpiperidine (98 mg, 0.2 mmol) in toluene (10 mL) was added, and the reaction mixture was stirred at room temperature for 11 days. The reaction mixture was distributed between brine and dichloromethane, and the organic layer was dried and evaporated to leave 190 mg of an oily residue. Radial chromatography (1 mm silica, dichloromethane followed by 2% methanol in dichloromethane) yielded 27 mg (17%) of 1-[6-(4-[(8-3-[2-(4-fluorophenyl)-1,3-dioxolan-2-yl]-propyl-4-phenyl-2,4,8-triazaspiro[4.5]dec-1-en-1-yl)-oxy]-methylpipetidino)-2-naphthyl]-1-ethanone (compound 3) and 92 mg (58%) of 1-(6-4-[(8-3-[2-(4-fluorophenyl)-1,3-dioxolan-2y-l]-propyl-1-oxo-4-phenyl-2,4,8-triazaspiro[4.5]dec-2-yl)-methyl]-piperidino-2-naphthyl)-1-ethanone (compound 4).

Compound 3: HRMS calcd. for C₄₃H₄₉FN4O₄: 704.3738. Found: 704.3760. ¹H NMR δ 1.46-1.90 (m, 10H, 3′a-H, 5′a-H, 3′e-H, 5′e-H, spiperone), 1.88 (m, 1H, 4′a-H), 2.15 and 2.38 (b, 4H, spiperone), 2.67 (s, 3H, COCH₃), 2.80 (m, 4H, spiperone), 2.95 (m, 2H, 2′a-H, 6′a-H), 3.75 (m, 2H, OCH₂CH₂O), 3.87 (m, 2H, 2′e-H, 6′e-H), 3.92 (m, 2H, OCH₂CH₂O), 4.19 (d, 2H, OCH₂), 4.97 (s, 2H, NCH₂N), 6.7-6.9 (m, 3H, Ph), 7.01 (m, 2H, Ph), 7.11 (d, 1H, 5-H), 7.23 (m, 211, Ph), 7.32 (dd, 1H, 7-H), 7.41 (m, 2H, Ph), 7.66 (d, 1H, 4-H), 7.81 (d, 1H, 8-H), 7.95 (dd, 1H, 3-H), 8.32 (d 1H, 1-H), J_{2′a,2′e}=J_{6′a,6′e}=12.4 Hz, J_{2′a,3′e}=J_{4′6,OCH2}=6.1 Hz, J_{1,3}=0.1 Hz, J_{3,4}=8.8 Hz, J_{5,7}=2.1 Hz, J_{7,8}=9.1 Hz.

Compound 4: HRMS calcd. for C₄₃H₄₉FN₄O₄:704.3738. Found: 704.3710. ¹H NMR δ 1.50 (dddd, 2H, 3′a-H, 5′a-H), 1.55-1.70 (m, 4H, spiperone), 1.84 (bd, 2H, 3′e-H, 5′e-H), 1.92 (m, 2H, spiperone), 1.98 (m, 1H, 4′a-H), 2.42 (m, 2H, spiperone), 2.67 (s, 3H, COCH₁), 2.69 (m, 2H, spiperone), 2.83 (m, 4H, spiperone), 2.88 (m, 2H, 2′a-H, 6′a-H), 3.35 (d, 2H, 4′-CH₂N), 3.75 (m, 2H, OCH₂CH₂O), 3.92 (bd, 2H, 2′e-H, 6′e-H), 4.02 (m, 2H, OCH₂CHO), 4.71 (s, 2H, NCH₂N), 6.88 (m, 1H, Ph), 6.91 (m, 2H, Ph), 7.01 (m, 2H, Ph), 7.08 (bs, 1H, 5-H), 7.27 (m, 3H, 7-H, Ph), 7.43 (m, 2H, Ph), 7.65 (d, 1H, 4-H), 7.79 (d, 1H, 8-H), 7.94 (dd, 1H, 3-H), 8.32 (bs, 1H, 1-H), J_{3′a,3′e}=J_{5′a,5′e}12.4 Hz, J_{2′a,3′a}=12.5 Hz, J_{2′a,2′e}=J_{6′a,6′e}=12.8 Hz, J_{2′a,3′e}=J_{6′a,5′e}=2.4 Hz, J_{4′a,4′}-CH₂N=7.3 Hz, J_{1,3}=1.9 Hz, J_{3,4}=8.8 Hz, J_{5,7}=2.1 Hz, J_{7,8}=9.2 Hz.

Using the procedure described in Example 1(b) for the synthesis of 2-[1-(6-ethyl-[2-(8-3-(2-(4-fluorophenyl)-1,3-dioxolan-2-yl]-propyl-1-oxo-4-phenyl-2,4,8-triazoaspiro[4.5]dec-2-yl)-ethyl]-amino-2-naphthyl)-ethylidene]-malonitrile, 1-(64[(8-3-[2-(4-fluorophenyl)-1,3-dioxolan-2y-l]-propyl-1-oxo-4-phenyl-2,4,8-triazaspiro[4.5]dec-2-yl)-methyl]-piperidino-2-naphthyl)-1-ethanone was transformed into 2-[1-(6-4-[(8-3-[2-(4-fluorophenyl)-1,3-dioxolan-2y-l]-propyl-1-oxo-4-phenyl-2,4,8-triazaspiro[4.5]dec-2-yl)-methyl]-piperidino-2-naphthyl)-ethylidene]malonitrile; It was purified by radial chromatography on a 1 mm silica plate using 2% MeOH in CH₂Cl₂ as the solvent. FAB HRMS calcd. for C₄₆H₅₀FN₆O₃ (M+H): 753.3928. Found: 753.3940. ¹H NMR δ 1.60-2.1 (m, 11H, spiperone, 3′a-H, 3′e-H, 4′a-H, 5′a-H, 5′e-H), 2.40 (m, 2H, spiperone), 2.71 (s, 3H, C═CCH₃), 2.60-2.80 (m, 6H, spiperone), 2.91 (m, 2H, 2′a-H, 6′a-H), 3.37 (d, 2H, 4′-CH2N), 3.75 (m, 2H, OCH₂CH₂O), 3.94 (bd, 2H, 2′e-H, 6′e-H), 4.02 (m, 2H, OCH₂CEO), 4.72 (s, 2H, NCH₂N), 6.85-6.95 (m, 3H, Ph), 7.01 (m, 2H, fluorophenyl), 7.07 (d, 1H, 5-H), 7.31 (m, 3H, 7-H, Ph), 7.41 (m, 2H, fluorophenyl), 7.56 (dd, 1H, 3-H), 7.69 (d, 1H, 4-H), 7.77 (d, 1H, 8-H), 8.01 (d, 1H, 1-H), J_{2′a,3′a}J_{5′a,6′a}=12.8 Hz, J_{2′a,2′e}=J_{2′a,2′e}=J_{6′a,6′e}=12.8 Hz, J_{4′a,4′-CH2N}=7.6 Hz, J_{1,3}=1.8 Hz, J_{3,4}=8.6 Hz, J_{5,7}=2.2 Hz, J_{7,8}=9.4 Hz, J_{2′a3′e}=J_{2′a,3′a}=J_{5′e,6′a}=1.8 Hz, J_{H,F}=8.7 and 6.2 Hz.

The ketal protective group was removed, as described in Example 1(b), to give 2-(1-6-[4-(8-[4-(4-fluorophenyl)-4-oxobutyl]-1-oxo-4-phenyl-2,4,8-triazaspiro[4.5]-dec-2-ylmethyl)-piperidino]-2-naphthylethylidene)-malononitrile in a quantitative yield. FAB HRMS calcd. for C₄₄H₄FN₆O₂ (M+H): 709.3666. Found: 709.3689.¹HNMR 1.60-2.1 (m, 11H, spiperone, 3′a-H, 3′e-H, 4′a-H, 5′a-H, 5′e-H), 2.5-2.71 (m, 4H, spiperone), 2.73 (s, 3H, C═C—CH₃), 2.8-3.1 (m, 6H, spiperone, 2′a-H, 6′a-H), 3.38 (d, 2H, 4′-CH₂N), 3.96 (bd, 2H, 2′e-H, 6′e-H), 4.74 (s, 2H, NCH₂N), 6.91 (m, 3H, phenyl), 7.1 (d, 1H, 5-H), 7.15 (m, 2H, fluorophenyl), 7.24-7.30 (m, 2H, Ph), 7.34 (dd, 1H, 7-H), 7.58 (dd, 1H, 3-H) 7.72 (d, 1H, 4-H), 7.79 (d, 1H, 8-H), 8.01-8.08 (m, 3H, fluorophenyl, 1-H). J_{1,3}=2.0 Hz, J_{3,4}=8.6 Hz, J_{5,7}=2.4 Hz, J_{7,8}=9.2 Hz, J_{4′a,4′-CH2N}=7.4 Hz, J_{2′a,2′e}=J_{6′a,6′e}=13.0 Hz, J_{2′a,3′a}=J_{5′a,6′a}=12.5 Hz, J_{2′a,3′e}=J_{5′e,6′a}=1.9 Hz, J_{H,F}=8.7 and 6.2 Hz.

### Example 1(d)

### Preparation of ten-Butyl-4-(6-acetyl-2-naphthyl)-1-piperazinecarboxylate

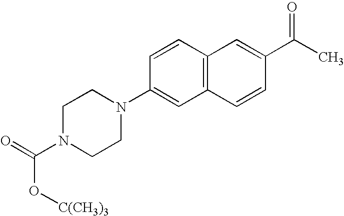

Anhydrous piperazine (7 g, 81.3 mmol; dried in a vacuum desiccator over KOH-drierite mixture for 3 days) was dissolved in a mixture of dry, freshly distilled toluene and hexamethyl phosphoric amide (HMPA), 25 mL each. To the solution was added 556 mg (80.1 mmol) of lithium rod, cut in small pieces under argon atmosphere, and the mixture was stirred under argon for 24 hours, during which time all lithium has dissolved. Vacuum-dried 1-(6-methoxy-2-naphthyl)-1-ethanone (prepared as described in Arsenijevic et al., Org. Synth. Coll. 1988 6:34-36, the disclosure of which is incorporated herein by reference) (3.5 g, 17.5 mmol) was added. and stirring was continued for additional 65 hours. After quenching with 300 mL of water, extraction with dichloromethane (3×300 mL), drying with anhydrous magnesium sulfate, and evaporation, a mixture of white and yellow solids was obtained. Extraction with 300 mL of hot methanol gave raw product, 1-(6-piperazino-2-naphthyl)-1-ethanone, which was purified by column chromatography (70-230 mesh silica, 25×120 mm, 5% methanol in dichloromethane). The yield was 1.54 g (35%). After recrystallization from ethyl acetate, the sample melted at 170.5-172° C.

1-(6-piperazino-2-naphthyl)-1-ethanone was also prepared by heating 1-(6-hydroxy-2-naphthyl)-1-ethanone (prepared as described in Example 1(b)) (441 mg, 2.36 mmol) at 140-150° C. with 6 g piperazine hydrate (30.9 mmol) and 244 mg (2.35 mmol) NaHSO₃ for 24 hours. Additional sodium bisulfite (2 g, 19.2 mmol) was added. After an additional 24 hours, more bisulfite (1 g) was added, and heating was continued (total reaction time 72 hours). After cooling, the mixture was extracted with 2×50 mL methanol. The residue after evaporation of methanol was suspended in 50 mL water and extracted with ethyl acetate (5×80 mL). Combined extracts were dried (magnesium sulfate) and evaporated to give 430 mg of yellow solid. Radial chromatography (4 mm silica, methanol) gave 83 mg (19%) of starting naphthol and 276 mg (46%; 56%, based on unrecovered starting material) of 1-(6-piperazino-2-naphthyl)-1-ethanone. The 1-(6-piperazino-2-naphthyl)-1-ethanone was in all respects identical with the compound obtained using the alternative method described above. Anal. calculated for C₁₆H₁₈N₂O: C, 75.56; H, 7.13; N, 11.01. Found: C, 75.82; H, 7.27; N, 10.92. ¹HNMR: δ 2.68 (s, 3H, CH₃), 3.09 and 3.35 (t, J=4.95 Hz, 8H, piperazine), 7.10 (d, 1H, 5-H), 7.31 (dd, 1H, 7-H), 7.69 (d, 1H, 4-H), 7.83 (d, 1H, 8-H), 7.95 (d, 1H, 3-H), 8.34 (s, 1H, 1-H); J_{5,7}=2 Hz, J_{7,8}=8.4 Hz, J_{1,3}=2 Hz, J_{3,4}=8.4 Hz.

1-(6-piperazino-2-naphthyl)-1-ethanone (254 mg, 1 mmol) was added to a stirred mixture of 1 g NaOH, 100 mg tetra-n-butylammonium hydrogensulfate, 2 mL water and 6 mL toluene, followed by a solution of 230 mg (1.05 mmol) of di-tert-butyl dicarbonate. The course of the reaction was followed by TLC (silica, 5% methanol in dichloromethane). Every 10 minutes an additional amount of the dicarbonate was added until all starting material had reacted. A total of approximately 1.5 equivalents were used. A mixture of water and dichloromethane (60 mL each) was added, and, after thorough shaking, the layers were separated. The aqueous layer was extracted with an additional 30 mL of dichloromethane. The combined organic extracts were dried with anhydrous magnesium sulfate. During this procedure, the color of the solution turned from pink to light yellow. Evaporation in vacuo gave 295 mg (83%) of ten-butyl-4-(6-acetyl-2-naphthyl)-1-piperazinecarboxylate, which, on recrystallization from dichloromethane-petroleum ether mixture, melted at 153-154° C. Anal. Calculated for C₂₁H₂₆N₂0₃: C, 71.16; H, 7.39; N, 7.90. Found: C, 71.27; H, 7.60; N, 7.86. ¹H NMR: δ 1.50 (s, 9H, —C(CH₃)₃), 2.68 (s, 3H, CH₃), 3.33 and 3.64 (t, J=4.9 Hz, 8H, piperazine), 7.10 (d, 1H, 5-H), 7.31 (dd, 1H, 7-H), 7.70 (d, 1H, 4-H), 7.85 (d, 1H, 8-H), 7.97 (d, 1H, 3-H), 8.35 (d, 1H, 1-H); J_{5,7}=2 Hz, J_{7,8}=9 Hz, J_{3,4}=8.7 Hz.

### Example 1(e)

### Preparation of 2-[1-(6-piperazino-2-naphthyl)ethylidene]malononitrile

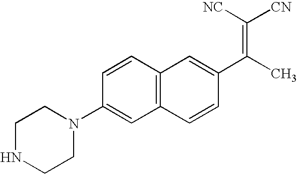

tert-Butyl 4-(6-acetyl-2-naphthyl)-1-piperazinecarboxylate (177 mg, 0.5 mmol), prepared as described in Example 1(d), was heated with 40 mg (0.6 mmol) of malononitrile in 4 mL pyridine at 105-110° C. After 5.5 hours, an additional 24 mg of malononitrile was added, and heating was continued for a total of 12 hours and 40 minutes. The mixture was cooled and evaporated in vacuo. Polar components of the mixture were removed by column chromatography (70-230 mesh silica, φ20×120 mm, chloroform), and the product, tert-butyl-4-[6-(2,2-dicyano-1-methylvinyl)-2-naphthyl]-1-piperazinecarboxylate, was finally purified by radial chromatography (silica, 2 mm, chloroform). 155 mg (77%) of tert-butyl-4-[6-(2,2-dicyano-1-methylvinyl)-2-naphthyl]-1-piperazinecarboxylate were obtained, which, after recrystallization from dichloromethane-petroleum ether mixture, melted at 169-171° C. Anal. Calculated for C₂₄H₂₆N₄O₂: C, 71.62; H, 6.51; N. 13.92. Found: C, 71.62; H, 6.66; N, 13.87. ¹H NMR: δ 1.50 (s, 9H, —C(CH₃)₃), 2.72 (s, 3H, CH₃), 3.34 and 3.64 (t, J=5.1 Hz, 8H, piperazine), 7.09 (d, 1H, 5-H), 7.33 (dd, 1H, 7-H), 7.58 (dd, 1H, 3-H), 7.74 (d, 1H, 4-H), 7.81 (d, 1H, 8-H), 8.02 (d, 1H, 1-H); J_{5,7}=2 Hz, J_{7,8}=9.1 Hz, J_{1,3}=2 Hz, J_{3,4}=9.1 Hz.

When tert-butyl-4-[6-(2,2-dicyano-1-methylvinyl)-2-naphthyl]-1-piperazinecarboxylate was treated with TFA (trifluoroacetic acid) at room temperature, TLC showed that the reaction was over in 5 minutes and gave a single product, 2-[1-(6-piperazino-2-naphthyl)ethylidene]malononitrile. The TFA was removed in vacuo at room temperature. ¹H NMR: δ 2.72 (s, 3H, CH₃), 3.50 and 3.63 (broad, 8H, piperazine), 7.18 (broad s, 1H, 5-H), 7.29 (d, 1H, 7-H), 7.59 (d, 1H, 3-H), 7.79 (d, 1H, 4-H), 7.87 (d, 1H, 8-H), 8.04 (s, 1H, 1-H), 9.0 (broad, 1.5H, NH and acid); J_{7,8}=8.8 Hz, J_{3,4}=8.4 Hz. ¹⁹F NMR: δ-76.2 (CF₃COO).

NMR of the residue revealed that the ten-butyloxycarbonyl group has been removed and that there was some TFA left (¹⁹F NMR). Dichloromethane (10 mL) was added and the solution was washed with saturated NaHCO₃ solution, dried, and evaporated in vacuo. A light yellow oil was obtained, which, on standing at room temperature, turned dark red. TLC showed that the change in color is due to decomposition of 2-[1-(6-piperazino-2-naphthyl)ethylidene]malononitrile into several products, the most intense spot being low-R_{f} red-orange. Selected ¹H NMR signals after neutralization: δ 2.72 (s, 3H, CH₃), 3.09 and 3.35 (t, J=5 Hz, 8H, piperazine), 7.08 (s, 1H, 5-H), 8.02 (s, 1H, 1-H).

### Example 1(f)

### Preparation of 2-(1,1-dicyanopropen-2-yl)-6-(2-[¹⁸F]-fluoroethyl)-methylamino)-naphthalene ([F-18]FDDNP)

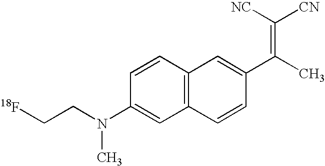

A mixture of 4.15 g (55.5 mmol) NaHSO₃, 8 mL of water, 0.78 g (4.19 mmol) of 1-(6-hydroxy-2-naphthyl)-1-ethanone (prepared as described in Example 1(b)), and 8 mL of 2-methylaminoethanol was heated and stirred in a steel bomb at 140° C. for 28 hours. After cooling, the mixture was distributed between ethyl acetate and water (500 mL and 200 mL, respectively). The organic layer was dried and evaporated to leave raw 1-(6-(2-hydroxyethyl-methylamino)-2-naphthyl)-1-ethanone (0.749 g, 73%) of which was further purified by radial chromatography (4 mm SiO₂, CH₂Cl₂).

To a solution of 201 mg (0.83 mmol) of 1-(6-2-hydroxyethyl-methylamino-2-naphthyl)-1-ethanone in pyridine (6 mL), malononitrile (236 mg, 3.6 mmol) was added and the mixture was heated at 95° C. for 24 hours. The solvent was removed in vacuo, and the residue was chromatographed by radial chromatography (4 mm SiO₂ 1% MeOH/CH₂Cl₂) to give 150 mg (73%) of 2-(1,1-dicyanopropen-2-yl)-6-2-hydroxyethyl)-methyl-amino)-naphthalene.

To the solution of 2-(1,1-dicyanopropen-2-yl)-6-(2-hydroxyethyl)-methylamino)-naphthalene (120 mg, 0.41 mmol) in pyridine (5 mL), p-toluensulfonic anhydride was added (441 mg, 1.35 mmol). After stirring at room temperature for 1 hour, pyridine was removed under vacuum, and the residue was chromatographed by radial chromatography (2 mm SiO₂, CH₂Cl₂) to give 183 mg (80%) of 2-(1,1-dicyanopropen-2-yl)-6-(2-tosyloxyethyl)-methylamino-naphthalene.

Radioactive ¹⁸F-fluoride 528.5 mCi from the cyclotron was transferred into a solution of 19 mg of Kryptofix 2.2.2 and 0.75 mg potassium carbonate in 50 μL of water and 300 μL of acetonitrile. Water was removed in a stream of nitrogen at 115° C. followed by codistillation with acetonitrile (3×200 μL). The tosylate (2-(1,1-dicyanopropen-2-yl)-6-(2-tosyloxyethyl)-methylamino)-naphthalene, 4 mg) in 1 mL of acetonitrile was added, and the mixture was heated at 85-86° C. for 20 minutes. After cooling, 1 mL of water was added, and the mixture was transferred onto a C-18 Sep-Pak Cartridge, washed with distilled water (3×4 mL) and eluted with CH₂Cl₂ (2×2.5 mL). Eluate was dried by passing through a column packed with sodium sulfate and loaded onto a HPLC column (Whatman Partisil Silica 10, 500×10 mm, mL/min CH₂Cl₂: hexane=7:3, UV detector @ 254 nm, radioactivity detector). Eluate was collected, appropriate fractions were combined, evaporated under vacuo to yield 50.7 m Ci (17%, corrected for decay) of the titled product which was formulated for injection. The synthesis was complete in 50 minutes.

### Example 1(g)

### Preparation of 7-(Dimethylamino)-2H-1-benzopyran-2-ones

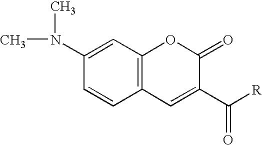

Vielsmeier reagent was prepared by addition of 4.66 mL (˜50 mmol) of POCl₃ (dropwise) to 7.76 mL of DMF at 0° C. in small aliquots, and the mixture was stirred for 5 min at the same temperature and then for 30 min at room temperature. 3.43 g (25 mmol) of 3-(dimethylamino)phenol in 2 mL of DMF was added to the Vielsmeier reagent dropwise. The mixture was heated at 60-70° C. for 1 hour, and the excess of reagent was destroyed by pouring the mixture on 50 g of crushed ice. The mixture was extracted with ethyl acetate, and the organic portion dried with anhydrous magnesium sulfate and evaporated. 4-dimethylamino-2-hydroxybenzaldehyde was isolated from the oily residue by column chromatography (silica gel, chloroform). Yield: 2.32 g (14 mmol, 56%).

4 mmol of 4-dimethylamino-2-hydroxybenzaldehyde and 5 mmol of the appropriate 1,3-dicarbonyl compound were dissolved in 8 ml of absolute ethanol. 16 drops of piperidine were added, and each mixture was heated under reflux for 2-6 hours. The mixtures were cooled in the freezer, and the precipitate was filtered off and washed with ethanol to provide the products set forth in Table 1.

TABLE 1

| | | React. | |
|---|---|---|---|
| R | 1,3-dicarbonyl compound | time | Yield |
| 1, —Me | ethyl acetoacetate | 6 h | 81% |
| 2, —OEt | diethyl malonate | 6 h | 40% |
| 3, —tBu | methyl 4,4-dimethylpentanoate | 2 h | 25% |
| 4, —CH₂—COOMe | dimethyl 1,3-acetonedicarboxylate | 6 h | 43% |
| 5, —Ph—NO₂ | methyl p-nitrobenzoylacetate | 2 h | 86% |

NMR spectra:
3-Acetyl-7-(dimethylamino)-2H-1-benzopyran-2-one(1)

¹H NMR (360 MHz, CDCl₃) δ: 2.70(3H, s, Ac); 3.14 (6H, s, NMe₂); 6.48 (1H, d, aromatic H); 6.66 (1H, dd, aromatic H); 7.43 (1H, d, aromatic H); 8.47 (1H, s, 4-H).
Ethyl 7-(dimethylamino)-2-oxo-2H-1-benzopyran-3-carboxylate(2)

¹H NMR (360 MHz, CDCl₃) δ: 1.40 (3H, t, EtO—); 3.13 (6H, s, NMe₂); 4.39 (2H, q, EtO—); 6.48 (1H, d, aromatic H); 6.65 (1H, dd, aromatic H); 7.40 (1H, d, aromatic H); 8.46 (1H, s, 4-H).
3-(2,2-dimethylpropanoyl)-7-(dimethylamino)-2H-1-benzopyran-2-one(3)

¹H NMR (360 MHz, CDCl₃) δ: 1.33 (9H, s, tBu); 3.10 (6H, s, NMe₂); 6.50 (1H, d, aromatic H); 6.63 (1H, dd, aromatic H); 7.33 (1H, d, aromatic H); 7.68 (1H, s, 4-H).
Methyl 3-[7-(dimethylamino)-2-oxo-2H-1-benzopyran-3-yl]-3-oxo-propanoate(4)

¹H NMR (360 MHz, CDCl₃) δ: 3.15 (6H, s, NMe₂); 3.75 (3H, s, —OMe); 4.11 (2H, s, CH₂); 6.47 (1H, d, aromatic H); 6.66 (1H, dd, aromatic H); 7.45 (1H, d, aromatic H); 8.53 (1H, s, 4-H).
7-(Dimethylamino)-3-(4-nitrobenzoyl)-2H-1-benzopyran-2-one(5)

¹H NMR (360 MHz, DMSO) δ: 3.13 (6H, s, NMe₂); 6.63 (1H, br, aromatic H); 6.84 (1H, dd, aromatic H); 7.69 (1H, d, aromatic H); 7.97 (2H, m, aromatic H); 8.31 (2H, m, aromatic H); 8.47 (1H, s, 4-H).

### Example 1(h)

### Preparation of Bis(7-(dimethylamino)-2-oxo-2H-1-benzopyran-3-yl)ketone

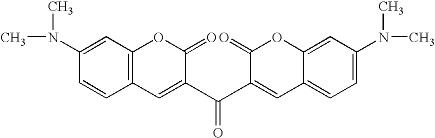

300 mg (1 mmol) of methyl 3-[7-(dimethylamino)-2-oxo-2H-1-benzopyran-3-yl]-3-oxo-propanoate [Compound 4 from Example 1(g)] was mixed with 250 mg of 4-dimethylamino-2-hydroxybenzaldehyde and dissolved in 2 mL of absolute ethanol. 4 drops of piperidine were added and the mixture refluxed for 8 hours. The mixture was brought to room temperature and the precipitated solid filtered off. 179 mg (44%) of an orange solid. ¹H NMR (360 MHz, CDCl₃) δ: 3.11 (6H, s, NMe₂); 6.50 (1H, d, aromatic H); 6.63 (1H, dd, aromatic H); 7.41 (1H, d, aromatic H); 8.19 (1H, s, 4-H).

### Example 1(i)

### Preparation of 2-{(2E)-3-(dimethylamino)-1-[6-(dimethylamino)-2-naphthyl]-2-propenylidene}malononitrile

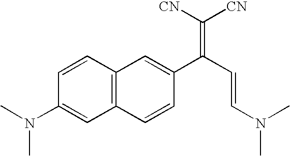

A solution of 2-{1-[6-(dimethylamino)-2-naphthyl]ethylidene}malononitrile (1 g, 3.8 mmol), prepared as described in A. Jacobson et al., J. Am. Chem. Soc. 1996, 118, 5572-5579, in N,N-dimethlyformamide dimethyl acetal (DMFDMA, 5 mL) was stirred at room temperature overnight. The volatiles were removed in vacuo at room temperature and the residue was chromatographed by column chromatography (silica 70-230 mesh, 20′ 150 mm, chloroform) to give 2-{(2E)-3-(dimethylamino)-1-[6-(dimethylamino)-2-naphthyl]-2-propenylidene}-malononitrile (1.19 g, 98%). mp 213-215° C. (from dichloromethane-petroleum ether mixture); Elemental analysis calculated for C₂₀H₂₀N₄: C, 75.92; H, 6.37; N, 17.17. Found: C, 76.28; H, 6.04; N, 17.57. IR (KBr pellet): 2210 cm⁻¹ (CN); ¹H NMR (360 MHz, CDCl₃) δ: 3.02 (s, 6H, Me₂N), 3.1 (s, 6H, Me₂N), 5.86 and 6.72 (d, 2H, CH═CH), 6.91 (d, 1H, H-5), 7.20 (dd, 1H, H-7), 7.23 (dd, 1H, H-3), 7.62 (bs, 1H, H-1), 7.69 (d, 1H, H-8), 7.73(d, 1H, H-4). J_{5,7}=2.3 Hz, J_{7,8}=9.1 Hz, J_{3,4}=9.0 Hz, J_{CH═CH}=12.5 Hz.

### Example 1(j)

### Preparation of 2-chloro-4-[6-(dimethylamino)-2-naphthyl]nicotinonitrile

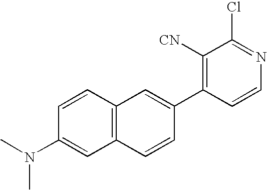

A solution of 2-{(2E)-3-(dimethylamino)-1-[6-(dimethylamino)-2-naphthyl]-2-propenylidene}malononitrile (603 mg, 1.9 mmol), prepared as described in Example 1(i) above, in isopropanol (100 mL) was saturated with dry HCl gas at rt and then stirred overnight. The solvent was removed in vacuo, the solid residue was dissolved in dichloromethane, and the solution was washed with saturated NaHCO₃ solution. The organic layer was dried and evaporated to leave 2-chloro-4-[6-(dimethylamino)-2-naphthyl]nicotinonitrile (402 mg, 69%). mp 198-200° C. (from MeOH); Elemental analysis calculated for C₁₈H₁₄N₃Cl: C, 70.24; H, 4.58; N, 13.65. Found: C, 69.76; H, 4.31; N, 13.38; IR (KBr pellet): 2250 cm⁻¹ (CN); ¹H NMR (360 MHz, CDCl₃) δ: 3.15 (s, 6H, Me₂N), 6.91 (d, 1H, H-5), 7.21 (dd, 1H, H-7), 7.47(d, 1H, H-5′), 7.57 (dd, 1H, H-3), 7.74 (d, 1H, H-8), 7.79 (d, 1H, H-4), 7.99 (d, 1H, H-1), 8.53 (d, 1H, H-6). J_{5,7}=2.2 Hz, J_{7,8}=9.7 Hz, J_{1,3}=2.0 Hz, J_{3,4}=9.2 Hz, J_{5′,6′}=5.1 Hz.

### Example 1(k)

### Preparation of 2-amino-4-[6-(dimethylamino)-2-naphthyl]nicotinonitrile

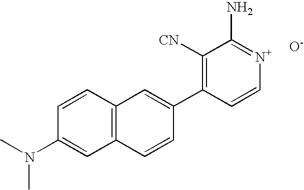

To a boiling solution of 2-{(2E)-3-(dimethylamino)-1-[6-(dimethylamino)-2-naphthyl]-2-propenylidene}malononitrile (100 mg, 0.32 mmol), prepared as described in Example 1(i) above, in MeOH (20 mL), ammonia gas was bubbled in for 30 min. Yellow crystals of 2-amino-4-[6-(dimethylamino)-2-naphthyl]nicotinonitrile, which started to separate from the solution during the reaction, were filtered off (86 mg, 93%). mp 256-257° C. (from MeOH); Elemental analysis calculated for C₁₈H₁₆N₄C, 74.98; H, 5.59; N, 19.43. Found: C, 75.29; H, 5.31; N, 19.16; IR (KBr pellet): 2210 cm⁻¹ (CN), 3270 cm⁻¹ (ArNH₂); ¹H NMR (360 MHz, CDCl₃) δ: 3.1 (s, 6H, Me₂N), 5.27 (s, 2H, NH₂), 6.87 (d, 1H, H-5′), 6.92 (d, 1H, H-5), 7.20 (dd, 1H, H-7), 7.57 (dd, 1H, H-3), 7.74 (d, 1H, H-8), 7.79 (d, 1H, H-4), 7.97 (bs, 1H, H-1), 8.42 (d, 1H, H-6); J_{5,7}=2.3 Hz, J_{7,8}=9.5 Hz, J_{1,3}=2.0 Hz, J_{3,4}=8.5 Hz, J_{5,6}=4.9 HZ.

### Example 1(l)

### Preparation of 2-amino-4-[6-(dimethylamino)-2-naphthyl]nicotinonitrile 1-oxide

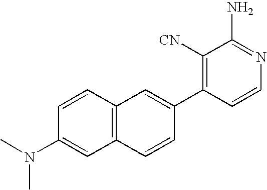

A solution of 2-{(2E)-3-(dimethylamino)-1-[6-(dimethylamino)-2-naphthyl]-2-propenylidene}malononitrile (286 mg, 0.9 mmol), prepared as described in Example 1(i) above, and hydroxyammonium chloride (94.5 mg, 1.36 mmol) in MeOH (20 mL) was heated under reflux for 70 h. The solvent was removed, and the residue was chromatographed by radial chromatography (2 mm silica, 5% MeOH in dichloromethane) to give 2-amino-4-[6-(dimethylamino)-2-naphthyl]nicotinonitrile 1-oxide (53 mg, 20%). mp 248-250° C. (from MeOH); Elemental analysis calculated for C₁₈H₁₆N₄O C, 71.04; H, 5.30; N, 18.41. Found: C, 70.59; H, 5.59; N, 18.19; IR (KBr pellet): 2220 cm⁻¹ (CN), 3310 cm⁻¹ (ArNH₂); ¹H NMR (360 MHz, CDCl₃) δ: 3.1 (s, 6H, Me₂N), 6.43 (s, 2H, NH₂), 6.86 (d, 1H, H-5′), 6.91 (d, 1H, H-5), 7.21 (dd, 1H, H-7), 7.55 (dd, 1H, H-3), 7.75 (d, 1H, H-8), 7.78 (d, 1H, H-4), 7.95 (bs, 1H, H-1), 8.12 (d, 1H, H-6); J_{5,7}=2.4 Hz, J_{7,8}=8.7 Hz, J_{1,3}=2.0 Hz, J_{3,4}=8.7 Hz, J_{5′,6′}=6.7 Hz.

### Example 1(m)

### Preparation of 4-[6-(dimethylamino)-2-naphthyl]-2-methoxynicotinonitrile

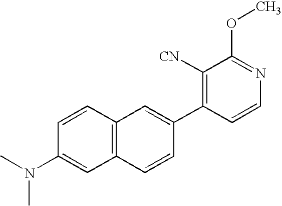

Method A. To a solution of 2-{(2E)-3-(dimethylamino)-1-[6-(dimethylamino)-2-naphthyl]-2-propenylidene}malononitrile (82.5 mg, 0.26 mmol), prepared as described in Example 1(i) above, in MeOH (15 mL), a solution of sodium methylate (1 mL, prepared by reacting 10 mg of sodium per mL of methanol) was added, and the reaction mixture was heated under reflux for 20 h. The solvent was removed in vacuo, the residue was distributed between dichloromethane and brine, and the organic layer was dried and evaporated. The residue was chromatographed by radial chromatography (1 mm silica, dichloromethane-cyclohexane 1:1) to yield 4-[6-(dimethylamino)-2-naphthyl]-2-methoxynicotinonitrile (40 mg, 50%).

Method B. A solution of 2-chloro-4-[6-(dimethylamino)-2-naphthyl]nicotinonitrile (58 mg, 0.19 mmol), prepared as described in Example 1(j) above, in MeOH (10 mL) was heated under reflux with sodium methylate (0.9 mL of solution, prepared by reacting 10 mg of sodium per mL of methanol, 0.4 mmol) for 6.5 h. The 4-[6-(dimethylamino)-2-naphthyl]-2-methoxynicotinonitrile was isolated as described under Method A but was purified by recrystallization from MeOH. The yield was 34 mg (60%). mp 150-151° C. (from MeOH); Elemental analysis calculated for C₁₉H₁₇N₃O C, 75.23; H, 5.65; N, 13.85. Found: C, 74.99; H, 5.59; N, 13.73; IR (KBr pellet): 2220 cm⁻¹ (CN); ¹H NMR (360 MHz, CDCl₃) δ: 3.1 (s, 6H, Me₂N), 4.11 (s, 3H, OMe), 6.9 (bs, 1H, H-5), 7.12 (d, 1H, H-5′), 7.20 (bd, 1H, H-7), 7.59 (d, 1H, H-3), 7.74 (d, 1H, H-4), 7.79 (d, 1H, H-8), 7.99 (bs, 1H, H-1), 8.31 (d, 1H, H-6′); J_{7,8}=9.2 Hz, J_{3,4}=8.6 Hz, J_{5′,6′}=5,4 Hz.

### Example 1(n)

### Preparation of 2-{(2Z)-1-[6-(dimethylamino)-2-naphthyl]-3-[(2-hydroxyethyl) amino]-2-propenylidene}malononitrile

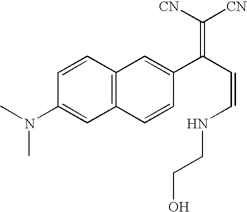

A solution of 2-{(2E)-3-(dimethylamino)-1-[6-(dimethylamino)-2-naphthyl]-2-propenylidene}malononitrile (350 mg, 1.1 mmol), prepared as described in Example 1(i) above, and 2-aminoethanol (0.4 mL, 6.6 mmol) in MeOH (50 mL) was heated under reflux for 24 h. The solvent was removed, and the residue was chromatographed by radial chromatography (4 mm silica, 5% MeOH in dichloromethane) to yield 2-{(2Z)-1-[6-(dimethylamino)-2-naphthyl]-3-[(2-hydroxyethyl)amino]-2-propenylidene}malononitrile (327 mg, 89%). mp 168-170° C. (from EtOH); ¹HNMR(360 MHz, CDCl₃) δ: 3.08 (s, 6H, Me₂N), 3.98 and 4.17 (bs, 4H, CH₂CH₂), 5.43 (b, 2H, OH, NH), 6.09 and 7.24 (d, 2H, CH═CH), 6.86 (bs, 1H, H-5), 7.16 (d, 1H, H-7), 7.51 (d, 1H, H-3), 7.68 (d, 1H, H-4), 7.74 (d, 1H, H-8), 7.94 (s, 1H, H-1); J_{3,4}=8.6 Hz, J_{7,8}=9.2 Hz, J_{CH═CH}=6.9 Hz.

### Example 1(o)

### Preparation of 4-[6-(dimethylamino)-2-naphthyl]-2-[(2-hydroxyethyl)sulfanyl]nicotinonitrile

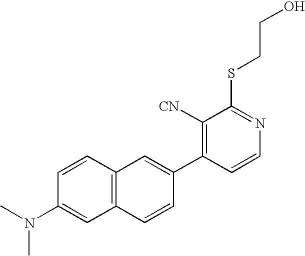

To a solution of 2-mercaptoethanol (0.81 mL, 11.6 mmol) in a solution of sodium methylate (5 mL, prepared by reacting 10 mg of sodium per mL of MeOH, 2.17 mmol), a solution of 2-chloro-4-[6-(dimethylamino)-2-naphthyl]nicotinonitrile (273 mg, 0.89 mmol), prepared as described in Example 1(j) above, in MeOH (20 mL) was added, and the mixture was heated under reflux for 5 h. The solid, which separated from the reaction mixture, was filtered off and recrystallized from acetonitrile to give 4-[6-(dimethylamino)-2-naphthyl]-2-[(2-hydroxyethyl)sulfanyl]nicotinonitrile (271 mg, 85%). mp 210-212° C.; Elemental analysis calculated for C₂₀H₁₉N₃OS: C, 68.74; H, 5.48; N, 12.02. Found: C, 68.51; H, 4.99; N, 12.07; IR (KBr pellet): 2220 cm⁻¹ (CN), 3350 cm⁻¹ (OH); ¹H NMR (360 MHz, CDCl₃) δ: 3.1 (s, 6H, Me₂N), 3.5 (t, 2H, CH₂CH₂), 3.65 (b, 1H, OH), 4.0 (b, 2H, CH₂CH₂), 6.91 (d, 1H, H-5), 7.20 (dd, 1H, H-7), 7.25 (d, 1H, H-5′), 7.55 (d, 1H, H-3), 7.75 (d, 1H, H-4), 7.79 (d, 1H, H-8), 7.97 (bs, 1H, H-1), 8.50 (d, 1H, H-6′); J_{7,8}=7.8 Hz, J_{3,4}=8.6 Hz, J_{5′,6′}=5.6 Hz, J_{CH2CH2}=5.4 Hz.

### Example 1(p)

### Preparation of 2-{(2Z)-3-[(2,2-diethoxyethyl)amino]-1-[6-(dimethylamino)-2-naphthyl]-2-propenylidene}malononitrile

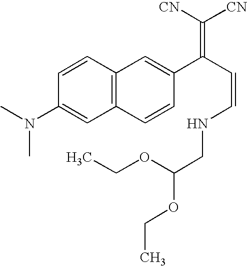

A solution of 2-{(2E)-3-(dimethylamino)-1-[6-(dimethylamino)-2-naphthyl]-2-propenylidene}malononitrile (100 mg, 0.316 mmol) and aminoacetaldehyde diethyl acetal (0.1 mL, 0.96 mmol) in MeOH (20 mL) was heated under reflux for 46 h. The solvent was removed in vacuo to leave an oily residue, which was chromatographed by radial chromatography (2 mm silica, dichloromethane, followed by 5% MeOH in dichloromethane) to yield 2-{(2Z)-3-[(2,2-diethoxyethyl)amino]-1-[6-(dimethylamino)-2-naphthyl]-2-propenylidene}malononitrile (111 mg, 87%). ¹H NMR (360 MHz, CDCl₃) δ: 1.22 (t, 6H, t, OCH₂CH₃, J=7.1 Hz), 3.09 (s, 6H, Me₂N), 3.59 and 3.80 (m, 4H, OCH₂CH₃), 4.05 (d, 2H, CH₂CH), 4.92 (t, 1H, CH₂CH), 5,95 and 7.23 (d, 2H, CH═CH), 6.90((bs, 1H, H-5), 7.19 (dd, 1H, H-7), 7.55 (d, 1H, H-3), 7.71 (d, 1H, H-4), 7.77 (d, 1H, H-8), 7.97 (bs, 1H, H-1); J_{CH═CH}=7.2 Hz, J_{7,8}=9.1 Hz, J_{3,4}=8.5 Hz, J_{CH2CH}=5.1 Hz.

### Example 1(g)

### Preparation of 2-{(2Z)-3-[(2,2-dimethoxyethyl)amino]-1-[6-(dimethylamino)-2-naphthyl]-2-propenylidene}malononitrile

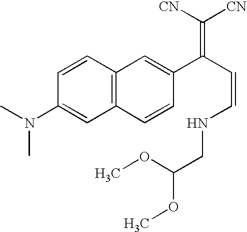

To a solution of 2-{(2Z)-3-[(2,2-diethoxyethyl)amino]-1-[6-(dimethylamino)-2-naphthyl]-2-propenylidene}malononitrile (19 mg, 0.05 mmol), prepared as described in Example 1(p) above, in MeOH (5 mL), HCl gas was bubbled in for 5 min at room temperature. The reaction mixture was stirred at room temperature for an additional 30 min and concentrated to leave an oily residue. It was distributed between ethyl acetate and ice-cold brine (40 mL each). The organic layer was dried and evaporated to yield 2-{(2Z)-3-[(2,2-dimethoxyethyl)amino]-1-[6-(dimethyl-amino)-2-naphthyl]-2-propenylidene}malononitrile (12 mg, 66%). 2-{(2Z)-3-[(2,2-dimethoxyethyl)amino]-1-[6-(dimethylamino)-2-naphthyl]-2-propenylidene}malononitrile was also prepared directly from 2-{(2E)-3-(dimethylamino)-1-[6-(dimethylamino)-2-naphthyl]-2-propenylidene}malononitrile, prepared as described in Example 1(i) above, using aminoacetaldehyde dimethyl acetal instead of the diethyl acetal and following the procedure for the synthesis of 2-{(2Z)-3-[(2,2-diethoxyethyl)amino]-1-[6-(dimethylamino)-2-naphthyl]-2-propenylidene}malononitrile as described in Example 1(p) above. mp 158-159° C.; IR (KBr pellet): 2220 cm⁻¹ (CN); ¹H NMR (360 MHz, CDCl₃) δ: 3.1 (s, 6H, Me₂N), 3.48 (s, 6H, OCH₃), 4.06 (d, 2H, CH₂CH), 4.78 (t, 1H, CH₂CH), 5,96 and 7.24 (d, 2H, CH═CH), 6.89 (d, 1H, H-5), 7.18 (dd, 1H, H-7), 7.54 (d, 1H, H-3), 7.74 (d, 1H, H-4), 7.76 (d, 1H, H-8), 7.96 (s, 1H, H-1); J_{5,7}=2.2 Hz, J_{CH═CH}=7.0 Hz, J_{7,8}=8.9 Hz, J_{3,4}=8.5 Hz, J_{CH2CH}=5.01 Hz.

### Example 1(r)

### Preparation of 7-[6-(dimethylamino)-2-naphthyl]imidazo[1,2-α]pyridine-8-carbonitrile

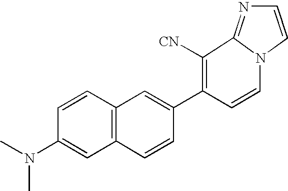

A solution of 2-{(2Z)-3-[(2,2-dimethoxyethyl)amino]-1-[6-(dimethylamino)-2-naphthyl]-2-propenylidene}malononitrile (53 mg, 0.14 mmol), prepared as described in Example 1(q) above, in MeOH (15 mL) was heated under reflux for 2 h. During the first 30 min a stream of dry HCl gas was led through the reaction mixture. After the reaction was complete, the solvent was evaporated and the solid residue was distributed between dichloromethane and saturated solution of sodium bicarbonate. The organic layer was dried and evaporated. The residue was cromatographed by radial chromatography (1 mm silica, 5% MeOH in dichloromethane) to give 7-[6-(dimethylamino)-2-naphthyl]imidazo[1,2-a]pyridine-8-carbonitrile (25 mg, 57%). mp 268-269° C.; Elemental analysis calculated for C₂₀H₁₆N₄: C, 76.90; H, 5.16; N, 17.94. Found: C, 76.231; H, 5.22; N, 17.62; IR (KBr pellet): 2230 cm⁻¹ (CN); ¹H NMR (360 MHz, CDCl₃) δ: 3.07 (s, 6H, Me₂N), 7.02 (d, 1H, H-5′), 7.29 (d, 1H, H-6), 7.33 (dd, 1H, H-7′), 7.69 (d, 1H, H-3′), 7.74 (s, 1H, H-3), 7.84 (d, 1H, H-4′), 7.87 (d, 1H, H-8′), 8.11 (s, 1H, H-1′), 8.14 (s, 1H, H-2), 8.9 (d, 1H, H-5); J_{5′,7′}=2.0 Hz, J_{7′,8′}, =9.4 Hz, J_{3′,4′}=8.6 Hz, J_{5,6}=7.2 Hz.

### Example 1 (s)

### Preparation of 4-[6-(dimethylamino)-2-naphthyl]-2-(methylamino)nicotinonitrle

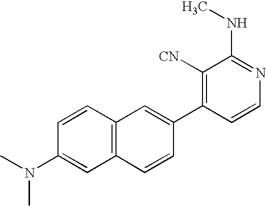

2-chloro-4-[6-(dimethylamino)-2-naphthyl]nicotinonitrile (194.5 mg, 0.63 mmol), prepared as described in Example 1(j) above, was suspended in ethanolic solution of methylamine (5 mL of 33% solution) and heated under reflux for 1 h. Additional 5 mL of methanolic methylamine was added and heating was continued for 9 h. After cooling, 4-[6-(dimethylamino)-2-naphthyl]-2-(methylamino)nicotinonitrile was filtered off (147 mg, 77%). Additional amount of product (40 mg), leading to a total yield of 98%, was isolated from the mother liquor by radial chromatography (1 mm silica, 1% of MeOH in dichloromethane). mp 193-194° C.; Elemental analysis calculated for C₁₉H₁₈N₄: C, 75.470; H, 6.00; N, 18.53. Pound: C, 75.28; H, 5.99; N, 18.48; IR (KBr pellet): 2210 cm⁻¹ (CN), 3270 cm⁻¹ (NH); ¹H NMR (300 MHz, CDCl₃) δ: 3.1 (s, 6H, NMe₂), 3.12 (d, 3H, MeNH, J=4.8 Hz), 5.38 (bs, 1H, NH), 6.76 (d, 1H, H-5′, J=5.3 Hz), 6.92 (d, 1H, H-5, J=2.5 Hz), 7.20 (dd, 1H, H-7, J=2.5 and 9.1 Hz), 7.56 (dd, 1H, H-3, J=1.9 and 8.6 Hz), 7.73 (d, 1H, H-4, J=8.6 Hz), 7.78 (d, 1H, H-8, J=9.1 Hz), 7.94 (d, 1H, H-1, J=1.9 Hz), 8.29 (d, 1H, H-6′, J=5.3 Hz).

### Example 1(t)

### Preparation of 7-(dimethylamino)-3-(4-fluorobenzoyl)-2H-1-benzopyran-2-one

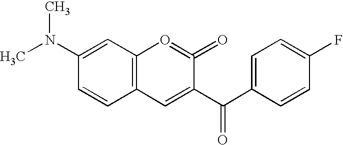

600 mg (1.6 mmol) of Kryptofix 2.2.2 and 87 mg (1.5 mmol) of potassium fluoride were dissolved in 1 mL of water and 3 mL of acetonitrile. The mixture was evaporated under a stream of argon at 100° C. 1 mL of acetonitrile was added, the mixture was evaporated, and this step was repeated twice. 170 mg (0.5 mmol) of 3-(4-nitrobenzoyl)-7-(dimethylamino)-2H-1-benzopyran-2-one, prepared as described in Example 1 (g) above, in 2 mL of anhydrous dimethyl sulfoxide was added, and the mixture was heated at 150° C. for 1 hour. The mixture was cooled and diluted with 10 mL of water, extracted with solid phase extraction (several C₁₈ Sep Pak cartridges), and eluted with dichloromethane, and the product was isolated by column chromatography with dichloromethane:methanol. 17 mg (0.5 mmol, 10%) of yellow solid. ¹H NMR (360 MHz, CDCl₃) δ:3.15 (6H, s, NMe₂); 6.53 (1H, d, aromatic H); 6.67 (1H, dd, aromatic H); 7.13 (2H, m, aromatic H); 7.41 (1H, d, aromatic H); 7.87 (2H, m, aromatic H); 8.14 (1H, s, 4-H). ¹⁹F NMR (CDCl₃) δ: −34.7 ppm

### Example 1(u)

### Preparation of 2-{1-[7-(dimethylamino)-2-oxo-2H-1-benzopyran-3-yl]ethylidene}malononitrile

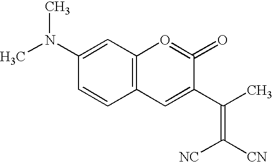

233 mg (1 mmol) of 3-acetyl-7-(dimethylamino)-2H-1-benzopyran-2-one, prepared as described in Example 1(g) above, and 70 mg (1 mmol) of malononitrile were dissolved, and 2 mL of pyridine and heated at 100° C. for 6 hours. The solvent was evaporated, and the product was isolated by column chromatography (silica gel, dichloromethane). 225 mg (81%) of an orange solid.

¹H NMR (360 MHz, CDCl₃) δ: 2.67(3H, s, Ac); 3.15 (6H, s, NMe₂); 6.51 (1H, br, aromatic H); 6.67 (1H, dd, aromatic H); 7.39 (1H, d, aromatic H); 7.95 (1H, s, 4-H).

### Example 1(v)

### Preparation of 7-[(2-hydroxyethyl)(methyl)amino]-2H-1-benzopyran-2-ones

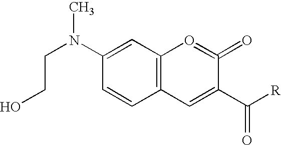

The synthetic approach for preparing 7-[(2-hydroxyethyl)(methyl)amino]-2H-1-benzopyran-2-ones is generally similar to the approach described for the dimethylamino analogs in Example 1(g) above. Because 3-[(hydroxyethyl)(methyl)aminophenol is not commercially available, a simple preparation method from resorcinol and the corresponding amine in the presence of boric acid as catalyst can be used. This synthetic procedure allows also preparation of N,N-disubstituted 3-aminophenols with the amino groups of choice.

30 g (272 mmol) of resorcinol, 25 mL (311 mmol) of 2-(methylamino)ethanol and 2.0 g (32 mmol) of boric acid were heated under reflux in a 3-neck flask equipped with a fractionation column. The water was distilled off and the internal temperature rose slowly from 180° C. to 230° C. over the course of 9 hours. After being cooled to approximately 60° C., 35 mL of methanol was added and the mixture of methyl borate and methanol was distilled slowly over the column. The excess of 2-(methylamino)ethanol was distilled at 15 mmHg, and the remaining oil was distilled under high vacuum (0.4 mmHg). A small amount of resorcinol distilled off between 155 and 165° C., and 3-[(hydroxyethyl)(methyl)amino]phenol was distilled off between 165-175° C. Yield: 32.5 g (215 mmol, 79%).

3-[(hydroxyethyl)(methyl)amino]phenol was added to Vielsmeier reagent (4 mol equivalent), prepared from POCl₃ and DMF at 0° C., and the mixture was heated at 60-70° C. for 1 hour. The reaction mixture was poured on ice and extracted with ethyl acetate. The organic portion was evaporated, and the residue was treated with diluted ammonia to de-formylate the hydroxyethyl group. After neutralization and extraction in ethyl acetate, the product was isolated with column chromatography (silica gel, chloroform).

To produce 7-[(2-hydroxyethyl)(methyl)amino]-2H-1-benzopyran-2-ones, 5 mmol of 4-[(hydroxyethyl)(methyl)amino]-2-hydroxybenzaldehyde and 6 mmol of the appropriate dicarbonyl compound were dissolved in 10 ml of absolute ethanol. 20 drops of piperidine were added and the mixture was heated under reflux for 2-6 hours. The mixture was cooled in the freezer and the precipitate filtered off and washed with ethanol.

### Example 1(w)

### Preparation of 2-(1-{7-[(2-hydroxyethyl)(methyl)amino]-2-oxo-2H-1-benzopyran-3-yl}ethylidene)malononitrile

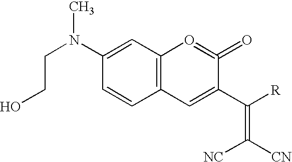

1 mmol of 3-acetyl-7-[(hydroxyethyl)(methyl)amino]-2H-1-benzopyran-2-one and 1 mmol of malononitrile were dissolved in 2 mL of pyridine and heated at 100° C. for 6 hours. The solvent was evaporated and the product was isolated by column chromatography (silica gel, dichloromethane).

### Example 1(x)

### Preparation of 2-[(3-acetyl-2-oxo-2H-1-benzopyran-7-yl)(methyl)amino]ethyl 4-methylbenzenesulfonate

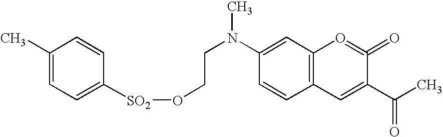

1 mmol of 3-acetyl-7-((hydroxyethyl)(methyl)amino]-2H-1-benzopyran-2-one was mixed with 3 mmol of p-toluenesulfonic anhydride in 2 mL of anhydrous pyridine at room temperature. After two hours the mixture was evaporated and the product was isolated by column chromatography (silica gel, ethyl acetate).

### Example 1(y)

### Preparation of 2-[[3-(2,2-dicyano-1-methylvinyl)-2-oxo-2H-1-benzopyran-7-yl](methyl)amino]ethyl 4-methylbenzenesulfonate

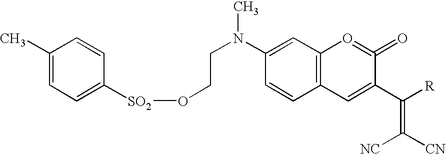

1 mmol of 3-acetyl-7-[(hydroxyethyl)(methyl)amino]-2H-1-benzopyran-2-one was mixed with 3 mmol of p-toluenesulfonic anhydride in 2 mL of anhydrous pyridine at room temperature. After two hours the mixture was evaporated and the product was isolated by column chromatography (silica gel, ethyl acetate).

### Example 1(z)

### Preparation of 3-acetyl-7-[(fluoroethyl)(methyl)amino]-2H-1-benzopyran-2-one

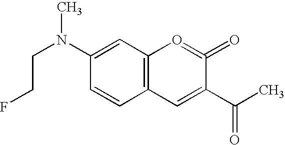

2 mmol of Kryptofix 2.2.2 and 2 mmol of potassium fluoride were dissolved in 1 mL of water and 3 mL of acetonitrile. The mixture was evaporated under a stream of argon at 100° C., re-dissolved in 1 mL of acetonitrile and evaporated (3 times). 1 mmol of 2-[(3-acetyl-2-oxo-2H-1-benzopyran-7-yl)(methyl)amino]ethyl 4-methylbenzenesulfonate in 2 mL of anhydrous acetonitrile was added and the mixture was heated at 90° C. for 20 min. The mixture was evaporated and the product isolated by column chromatography.

### Example 1(aa)

### Preparation of 2-(1-{7-[(2-fluoroethyl)(methyl)amino]-2-oxo-2H-1-benzopyran-3-yl}ethylidene)malononitrile

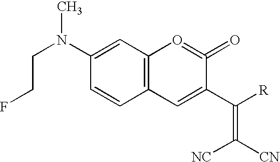

2 mmol of Kryptofix 2.2.2 and 2 mmol of potassium fluoride were dissolved in 1 mL of water and 3 mL of acetonitrile. The mixture was evaporated under a stream of argon at 100° C., redissolved in 1 mL of acetonitrile and evaporated (3 times). 1 mmol of 2-[[3-(2,2-dicyano-1-methylvinyl)-2-oxo-2H-1-benzopyran-7-yl](methyl)amino]ethyl 4-methylbenzenesulfonate in 2 mL of anhydrous acetonitrile was added and the mixture was heated at 90° C. for 20 min. The mixture was evaporated and the product isolated by column chromatography.

### Example 2

Detection and labeling of β-amyloid plaques in vitro and in vivo, using brain tissue sections and rat brains, were conducted using the following procedures.

A 2.1 mg/mL DDNP stock solution was prepared, which was adjusted to 8 mM in 100% ethanol. A DDNP working solution was prepared by diluting the stock solution with distilled water in a ratio of 1:100-1000 (stock solution:distilled water).

β-amyloid 250 μM (1.25 mg/mL in distilled water) was aggregated at 37° C. for 48 hours. 5 μL were smeared on slides, air-dried and then rehydrated with distilled water. Alternatively, Aβ-positive brain tissue sections were rehydrated with distilled water. DDNP working solution was applied to each slide for 30 minutes at room temperature. The slides were washed three times for five minutes with distilled water. The slides were coverslipped with fluorescent protectant mounding media (Vectashield™, available Vector Labs., Burlingame, Calif.) and observed under a fluorescence microscope with a thioflavin S or FITC filter.

β-amyloid 250 μM (1.25 mg/mL in distilled water) was aggregated at 37° C. for 48 hours to produce fibrils confirmed by smears. Three rats were anesthetized. 3 μL of a solution of Aβ fibrils (1.25 μg/μL) were injected unilaterally into the cortex of each rat (Bregma 0, AP-4.1 mm, ML+2.0 mm, DV-3.1 mm). Then 3 μL of phosphate buffered saline (PBS) were injected into the contralateral side of each rat brain as a vehicle control. After injection, the needle remained for 5 minutes to prevent reflux, and then the cranial hole was sealed with bone wax. Eight days after β-amyloid injection into the rat brains, the rats were injected with 10 microliters of DDNP working solution (320 micromolar) prepared by diluting DDNP stock solution into 1.5% BSA (bovine serum albumin) in phosphate buffered saline, pH 7.2).

After one hour, the rats were cardiac perfused with PLP fixative (4% paraformaldehyde, 1% lysin in 0.05 M phosphate buffer, pH 7.4). Additional immersion fixation of rat brain was at 4° C. overnight with PLP fixative. The rat brains were washed with PBS, saturated in 10 and 20% sucrose, and snap frozen in chilled isopentane (−70° C.) with liquid nitrogen. The brains were cryostat sectioned at 10 μM around the needle-track and directly coverslipped with glycerol and fluorescence protectant (Vectashield™). The brain sections were observed with a fluorescence microscope.

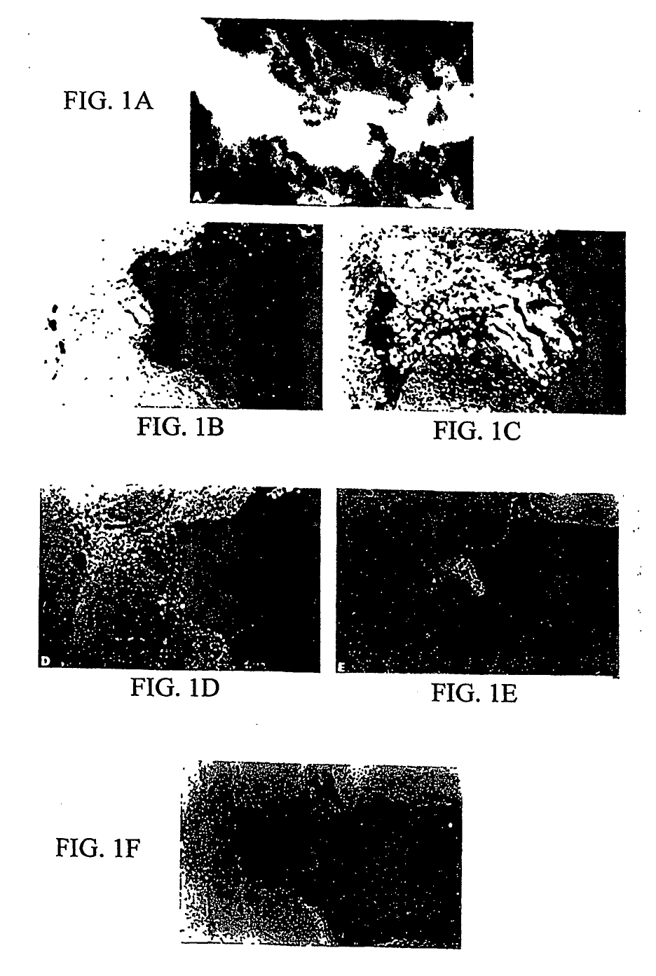

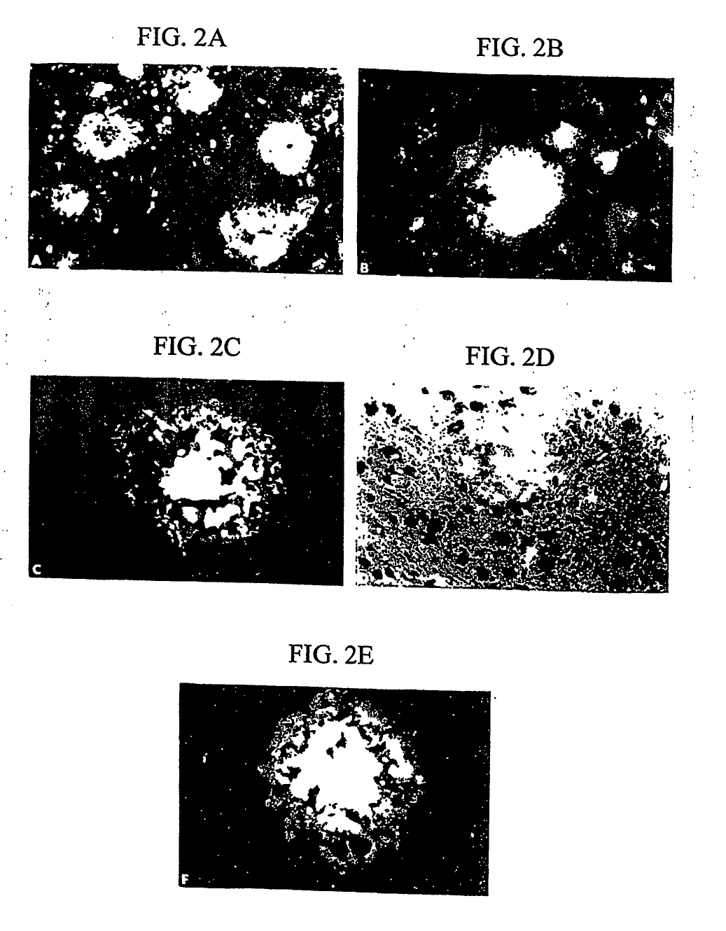

FIGS. 1A to 1F depict amyloid plaques labeled in sections from brain of an AD patient and a transgenic mouse, demonstrating that DDNP is able to label amyloid plaques. FIGS. 2A to 2E depict labeled β-amyloid plaques, demonstrating that DDNP passes the blood brain barrier in rats.

It was found that DDNP readily labeled amyloid deposits in cryostat and paraffin sections of AD brain tissue with a level of sensitivity similar to thioflavin S. Use of DDNP has several advantages over thioflavin S. Namely, the use of DDNP requires no pretreatments and, unlike thioflavin S, works with minimal washing and without formalin or paraformaldehyde fixation or differentiation of tissue. Stock solution can be kept in the freezer for six months and still produce acceptable results at 1/100 to 1/1,000 dilutions, eliminating the need to make the stock up fresh, as is required for thioflavin S labeling.

### Example 3

Labeling of human β-amyloid plaques and neurofibrillary tangles in vivo were conducted using the following procedures.

A patient was placed in a tomograph to obtain brain dynamic PET images. 8.0 mCi of 2-(1.1-dicyanopropen-2-yl)-6-(2-[¹⁸F]-fluoroethyl)-methylamino)-naphthalene ([F18]FDDNP) (specific activity: 5-12 Ci/micromol; mass: ˜1 nanomol), prepared as described in Example 1(f), were injected intravenously into the arm of the patient. Dynamic acquisition data of brain images were recorded simultaneously in forty-seven brain planes for two hours.

It was found that [F-18]FDDNP readily crosses the brain blood barrier and labels brain structures in a manner consistent with the presence of beta amyloid plaques and neurofibrillary tangles. The patient had previously had ¹⁸F-fluorodeoxyglucose (FDG)/positron emission tomography (PET) scans, as well as MRI scans to monitor brain atrophy. In areas where the maximum atrophy was observed in the MRI scans (low temporal and parietal lobes), maximum accumulation of the [F-18]FDDNP label was observed. In those areas, low glucose metabolism (as measured with FDG/PET scans) was also observed.

### Example 4

Labeling and detection of human β-amyloid plaques and neurofibrillary tangles in vivo were conducted using the following procedures. Ten human subjects, seven Alzheimer's diseased patients (ages 71 to 80) and three control patients (ages 62 to 82) were studied. The patients were positioned supine in an EXACT HR+962 tomograph (Siemens-CTI, Knoxville, Tenn.) with the imaging plane parallel to the orbito-meatal line. Venous catheterization was performed, and then [F-18]FDDNP (5-10 mCi) in human serum albumin (25%) was administered as a bolus via the venous catheter. Sequential emission scans were obtained beginning immediately after [F-18]FDDNP administration using the following scan sequence: six 30 second scans, four 3 minute scans, five 10 minute scans, and three 20 minute scans. Rapid venous blood sampling was performed via the indwelling catheter in two subjects for input function determination and plasma metabolite analysis.

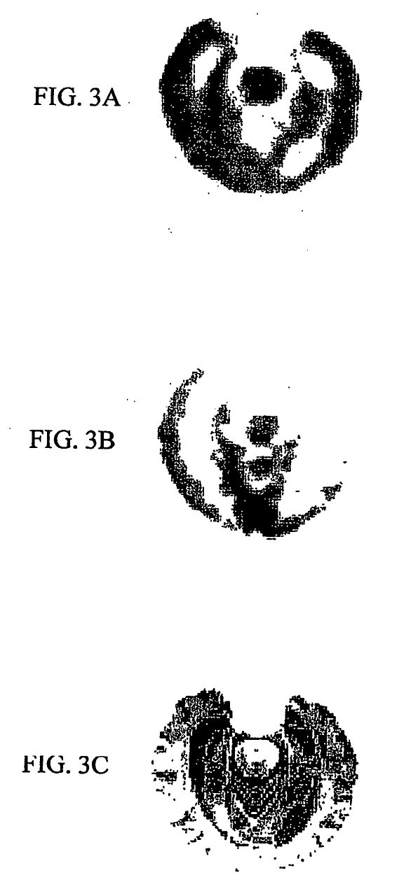

FIG. 3A provides a PET-[F-18]FDDNP (2-(1.1-dicyanopropen-2-yl)-6-(2-[¹⁸F]-fluoroethyl)-methylamino)-naphthalene) image of a brain cross-section through the hippocampus-amygdala-entorhinal/temporal cortex region of an Alzheimer's disease patient. The image was reconstructed from scanning data obtained 30 to 60 minutes post [F-18]FDDNP injection. Co-registered PET-FDG (FDG is 2-[F-18]fluoro-2-deoxy-D-glucose) and MRI (proton relaxation times) images of the patient are also shown, in FIGS. 3B and 3C, respectively, to provide information about glucose metabolism and anatomical structures on the cross-section, respectively. The medial temporal region appears darker in the [F-18]FDDNP scan (slower clearance) and lighter in the FDG scan (reduced glucose metabolism).

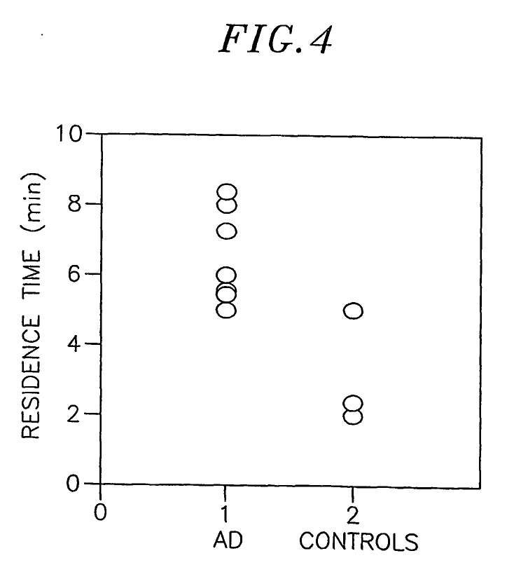

FIG. 4 demonstrates that estimated residence times of [F-18]FDDNP in Alzheimer's diseased patients are seen to be different from values in control patients. The residence time shown is in reference to that in the pons, an area known to have limited involvement in Alzheimer's disease pathology. The residence time was calculated from the clearance rates of the tracer in affected regions of interest (ROI) and in the pons as:
Residence time=[1/clearance rate for affected ROI]−[1/clearance rate for pons]
Separate ROIs were defined in entorhinal cortex, hippocampus, lateral temporal cortex and pons. The region with the slowest clearance rate was used as the affected ROI in the calculation of the residence time shown in FIG. 4.

It was found that after intravenous injection, [F-18]FDDNP crosses the blood brain barrier readily in proportion to blood flow. Accumulation of radioactivity was followed by the differential regional clearance of [F-18]FDDNP. A slower clearance was observed in brain areas reliably known to accumulate β-amyloid plaques and neurofibrillary tangles, specifically the hippocampus-amygdala-entorhinal complex, as well as temporal and parietal cortex in more advanced states of the disease. rCMRG1 measured with PET in these subjects were also consistent with the expected β-amyloid plaque load and the possible presence of neurofibrillary tangles. In these patients, brain areas with low glucose metabolism were in general matched with high retention of [F-18]FDDNP. The hippocampus-amygdala-entorhinal cortex presented high retention of activity ([F-18]FDDNP) in most cases, even in patients with low severity of symptoms. A normal 82 year old volunteer presented deposition of activity in the hippocampus-amygdala-entorhinal complex in a PET study with [F-18]FDDNP, and low rCMRG1 in the same areas, as measured with FDG, as shown in FIG. 4. These results are consistent with observations that elderly individuals without apparent signs of dementia may present neurofibrillary pathology in the second layer of neurons of the entorhinal cortex and plaques in the hippocampal formation. Increased severity of symptoms was always accompanied with increased retention of activity, and slow clearance from temporal, parietal or frontal cortex, in agreement with expected Aβ and neurofibrillary tangles deposition in these areas.

In vitro autoradiography using (F-18]FDDNP with brain specimens of Alzheimer diseased patients also demonstrated a distribution of activity consistent with the presence of β-amyloid plaques and neurofibrillary tangles. Binding was observed in hippocampus, temporal and parietal cortex matching results with immunostaining Aβ and tau antibodies. Since DDNP and its derivatives are fluorescent, an evaluation of the ability of [F-18]FDDNP to label β-amyloid plaques and neurofibrillary tangles in vitro was also performed with the same brain specimens. In all Alzheimer's disease brain specimens, excellent visualization of neurofibrillary tangles, amyloid peptides, and diffuse amyloid was produced with both DDNP and [F-18]FDDNP, matching results with thioflavin S (24) obtained with the same samples.

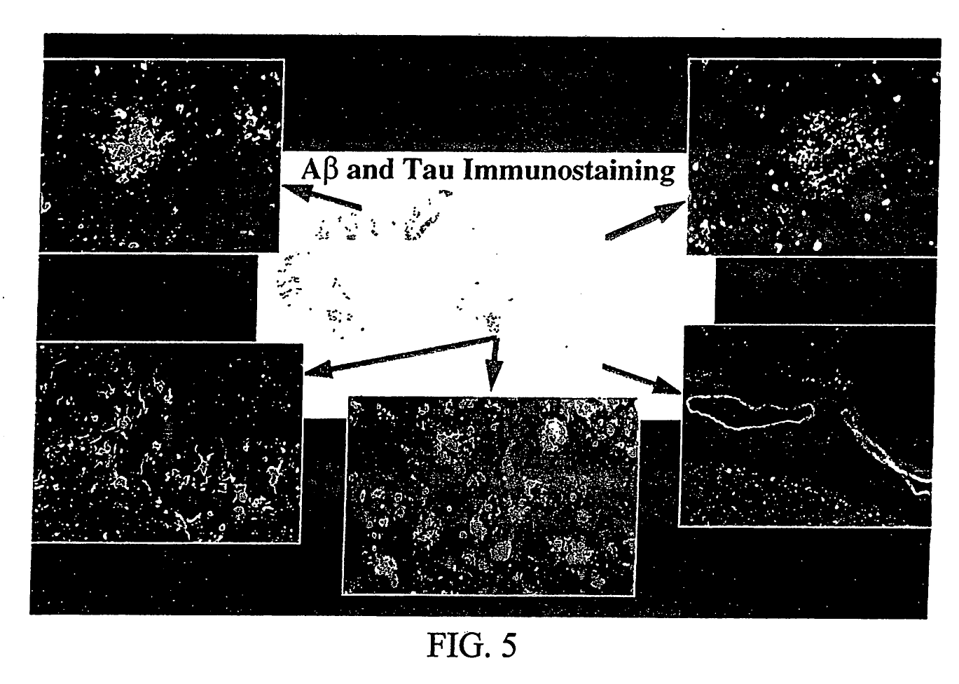

In FIG. 5, the central image was obtained by immunostaining a forty five micrometer cryostate temporal cortex section of an Alzheimer's disease patient incubated with AT8 (anti-phosphotau) and 10G4 (anti-AB1-15) at 1:800. Insets are adjacent sections of the same Alzheimer's disease brain specimen stained with FDDNP. Images were generated using fluorescent scanning microscopy. Green arrows indicate approximate origin of inset with reference to central immunostaining section. Beginning in upper left corner and moving clockwise, the insets show (1) neuritic plaques, (2) diffuse plaque, (3) vascular amyloid, (4) dense plaques and tangles, and (5) dense tangles.

### Example 5

Potential therapeutic agents were evaluated to determine their ability to bind to β-amyloid fibrils.
Amyloid (1-40) fibril formation.
   β-amyloid (1-40) (Biosource, Camarillo, Calif.) fibrils were prepared. 0.5 mg of β-amyloid (1-40) was dissolved in 1 mL PBS, pH 7.4 and mixed with a magnetic stir bar for 3 days at 37° C. resulting in a visibly cloudy solution. Fibrils were used immediately after their production was confirmed. The production of 13-amyloid fibrils was confirmed by imaging with a Jeol 100CX transmission electron microscope (Jeol, Peabody, Mass.). A 5 μL drop from a fibril solution was allowed to settle for 30 s on a treated copper grid before being washed away with a drop of 2% uranyl acetate solution. Finally, an additional drop of 2% uranyl acetate was added to the grid to negatively stain the fibrils. Fibrils were used immediately after their production was confirmed. Additional tests for fibril formation using Congo red (CR, Sigma) and Thioflavine T (TT, Sigma) were performed, as well. The absence of fibrils in the filtrate in the in vitro competition assays described below was determined by the same tests using CR.
   In vitro competition assays with [¹⁸F]FDDNP against NSAIDs and charge amyloid dyes. Solutions of nonradioactive agents, namely (S)-naproxen (Sigma), (R)-ibuprofen (BIOMOL, Plymouth Meeting, Pa.), (S)-ibuprofen (Sigma), diclofenac (Sigma), CR and TT, were prepared fresh for each radioactive competition assay. APFF glass fiber filters (0.7 μm particle retention; Millipore, Bedford, Mass.) were used in a 1225 sampling manifold (Millipore) that was modified with stainless steel support screens (Millipore) and glass sample chambers. Vacuum filtration involved 0.865 μg/mL of in vitro fibrils of synthetic β-amyloid (1-40) and 37 MBq/mL of [¹⁸F]FDDNP incubated for 1 h in PBS, pH 7.4 (1% ethanol) with various concentrations of the nonradioactive agents with the range of 0.1 pM to 83 μM. Each filter was then washed twice with 3 ml of PBS, pH 7.4. The radioactivity retained by the filters was measured and decay-corrected to a common reference time with a Packard Cobra II Auto-Gamma gamma counter (Packard, Meriden, Conn.). The binding of radiolabeled [¹⁸F]FDDNP to β-amyloid fibrils without competitor determined 100% specific binding. It was assumed that 100% competition against radioactive [¹⁸F]FDDNP occurred with 40 μM of nonradioactive FDDNP. All competition assays were performed in triplicate. Competition results were analyzed and then Kᵢ values calculated using the Ligand Binding Module for SigmaPlot 2001 (SPSS, Chicago, Ill.).
   Preparation of Brain Tissue.

Brain specimens from a 79 year-old female postmortem-diagnosed definite AD patient were treated. Briefly, formalin-treated, cryoprotected brain specimens were sectioned 70 μm thick coronally mounted on gelatine-coated glass slides, allowed to dry, and were defatted for 40 min in xylene prior to rinsing of the tissue with ethanol. Finally, lipofuscin autofluorescence in some brain specimens was quenched prior to staining using 10 mM CuCl₂ in 50 mM ammonium acetate buffer, pH 5. The quenching determined the origin of lipofuscin fluorescence in brain specimens.

Digital Autoradiography

Post-mortem diagnosed definite AD brain specimens (70 μm thick) were pretreated with either 100 nM of fresh batches of nonradioactive FDDNP and (S)-naproxen or 40 μM of (R)-ibuprofen, (S)-ibuprofen, diclofenac, CR or TT in 10% ethanol in PBS, pH 7.4, for 60 min and then the liquid decanted prior to digital autoradiography with [¹⁸F]DDNP. Pretreated and cryosections with no competitor were incubated for 25 min at room temperature with 3.7 GBq of [¹⁸F]FDDNP dissolved in 10 mL of 1% ethanol in 0.9% saline (w/v) per cryosection. Following incubation, the sections were optimally washed with water (30 sec); 60% 2-methyl-2-butanol (3 min; Sigma) agitated at 40 RPMs on a Junior Orbit Shaker (Lab-Line Instruments, Melrose Park, Ill.) for differentiation (Bancroft and Stevens, 1990); and then water (30 sec). The sections were dried on a warm hot plate with a steady stream of warm air, exposed to β⁺-sensitive phosphor plates for 40 min Fuji Film Medical Systems USA, Stamford, Conn.), and scanned with a FUJI BAS 5000 Phosphorimager (Fuji) at a resolution of 25 μm, as described previously (Agdeppa et al., 2001a). Radioactivity in tissue scrapings from the imaged specimens were subsequently measured in a Packard Cobra II Auto-Gamma (Packard), decayed to common reference time, and used as radioactive standards to quantify the amount of specific binding of [¹⁸F]FDDNP (Radioactivity/Area, Bq/mm²; FIG. 2Q) in the autoradiograms. Autoradiography was carried out at least in triplicate for each competitor. Statistical analysis of the autoradiograms involved one-way ANOVA with Dunnett's post test using Prism 3.02 (GraphPad, San Diego, Calif.) to compare the differences in the ratio of gray matter-to-white matter (FIG. 2Q) radioactivity of [¹⁸]FDDNP in autoradiograms pretreated with the nonradioactive agents and without pretreatment. Unpaired t-tests were performed to compare the difference of measured [¹⁸F]FDDNP radioactivity per area of tissue (Bq/mm²) in the gray and white matter of each autoradiogram.

Fluorescence Microscopy.

The same brain specimens used for autoradiography were observed using fluorescence microscopy. Tissues were mounted with Vectashield (Vector, Burlingame, Calif.) and observed with a Nikon Labophot fluorescence microscope (Nikon USA, Melville, N.Y.) with a FITC filter set.

Fluorescence microscopy of tissue previously used for autoradiography with [¹⁸F]FDDNP is possible due to the fluorescent properties of FDDNP (Jacobson et al., 1996) and the labeling of SPs by residual nonradioactive FDDNP. The specific activity (activity per unit mass) of non-carrier-added [¹⁸F]FDDNP at the end of synthesis was 74222 GBq/μmol (2000-6000 Ci/mmol), 10³ times lower than the maximum theoretical specific activity (Sorenson and Phelps, 1987). Thus, after ¹⁸F decay the residual nonradioactive FDDNP bound to SPs in AD brain specimens may be imaged with fluorescence microscopy.

The above tests demonstrated that naproxen and ibuprofen share the same binding sites of [^{˜}F]FDDNP on β-amyloid fibrils. In vitro radioactive competition curves carried out using various concentrations of non-radioactive agents co-incubated with [¹⁸F]FDDNP and synthetic β-amyloid (1-40) fibrils revealed one site-binding competition for (S)-naproxen, (R)-ibuprofen, and (S)-ibuprofen (p=0.05). The concentration dependent decrease in the binding of [¹⁸]FDDNP versus (S)-naproxen, (R)-ibuprofen, and (S)-ibuprofen yielded Kᵢ values of Kᵢ=5.70 ±1.31 nM (±SD), Kᵢ=44.4±17.4 μM (±SD), and Kᵢ₌11.3±5.20 μM (±SD), respectively, indicating that (S)-naproxen binds more tightly to β-amyloid. Diclofenac, CR, and TT did not exhibit a dose dependent decrease in the specific binding of [¹⁸F]FDDNP.

Further, autoradiography with naproxen and ibuprofen demonstrated complete blockade of [¹⁸F]FDDNP binding sites on ex vivo senile plaques. The gross pattern of radioactivity in the coronal AD brain specimens with no competitor revealed the specific binding of [¹⁸F]FDDNP to areas containing senile plaques. Specific binding of [¹⁸F]FDDNP to regions of gray matter with senile plaques was significantly reduced in AD specimens pretreated with nonradioactive FDDNP, (S)-naproxen, (R)-ibuprofen, and (S)-ibuprofen compared with autoradiography in the absence of those competitors. With these competitors the difference in the radioactivity (Radioactivity/Area, Bq/mm²; FIG. 2Q) between senile plaque-laden gray matter areas and white matter void of senile plaques was not significant (p>0.05) and was consistent with the significantly lower (p<0.05) gray matter-to-white matter ratio of radioactivity in comparison to the ratio for the [¹⁸F]FDDNP autoradiograms with no competitor. These results indicate blockage of specific binding of [¹⁸F]FDDNP down to background levels of radioactivity found in the white matter. All these results were confirmed by fluorescence microscopy with the same brain specimens. Diclofenac, CR and TT had minimal effect on specific binding of [¹⁸F]FDDNP to senile plaques as determined by autoradiography and fluorescence microscopy of the same tissue specimens. The difference between gray and white matter radioactivity was significantly different (p<0.05) for diclofenac, CR, and IT, as was the case for [¹⁸F]FDDNP autoradiography with no competitor. The comparison of the ratios of gray matter-to-white matter radioactivity between diclofenac, CR, and TT with [¹⁸F]FDDNP with no competitor indicate the preservation of specific binding of [¹⁸F]FDDNP to senile plaques.

More details on the methods of the invention are provided in Appendix A and Appendix B hereto, the disclosures of which are incorporated herein by reference.

This invention in its broader aspect is not limited to the specific details shown and described herein. Departures from such details may be made without departing from the principles of the invention and without sacrificing its chief advantages.

We claim:

1. A method for labeling structures selected from the group consisting of β-amyloid plaques and neurofibrillary tangles either in vivo or in vitro, comprising contacting brain tissue with a compound of formula (IA) or (IB): 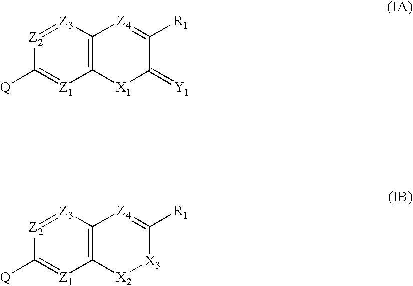
wherein:
Q is selected from the group consisting of —NR₂R₃, —OR₉ and —SR₉;
X₁ is selected from the group consisting of O, S, —NR₄, —CH₂, —CH-alkyl, and —C(alkyl)₂;
X₂ is selected from the group consisting of N and —CR₉;
X₃ is selected from the group consisting of N and —C—Y₂;
Y₁ is selected from the group consisting of O, S, —NR₉, and —C(R₉)₂;
Y₂ is selected from the group consisting of R₉, —N(R₉)₂—OR₉ and —SR₉;
Z₁, Z₂, Z₃, and Z₄ are each independently selected from the group consisting of N and —CR₉;
R₁ is selected from the group consisting of —CN, —C(=A₁)-A₂, alkyl-A₂, alkenyl-A₂, alkynyl-A₂, 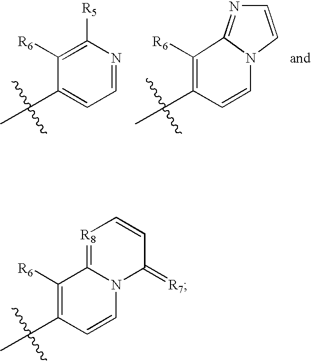
wherein:
A₁ is selected from the group consisting of O, S, —NR₄, —C(CN)₂, —C(CN)COOR₉, and —C(COOR₉)₂, and
A₂ is selected from the group consisting of OH, —O-alkyl, —NH₂, —NH—R₄, —N(R₄)₂, halogen, alkyl, cyclic ring, heterocyclic rings, —O-alkylenyl-R₄, —NH-alkenyl —R₄, -alkenyl-R₄, -alkenyl-NHR₄, -alkenyl-NH₂, -alkenyl-N(R₄)₂, —O-alkenyl-R₄, —NH-alkynyl-R₄, -alkynyl-R₄, -alkynyl-NHR₄, -alkynyl-NH₂, -alkynyl-NR₄)₂,
or A₁ and A₂ together form a cyclic ring or heterocyclic ring,
or R₁ together with X₃ or Z₄ forms a cyclic ring or heterocyclic ring;
R₄ is a radical selected from the group consisting of hydrogen, alkyl, alkenyl, alkynyl, cyclic rings, heterocyclic rings, —C(O)O-alkyl, —C(O)O-alkenyl, —C(O)O-alkynyl, OH, OTs, and halogen;
R₅ is a radical selected from the group consisting of hydrogen, alkyl, alkenyl, alkynyl, cyclic rings, heterocyclic rings, —OH, —OTs, —SH, halogen, —N(R₄)₂, —O-alkyl, —O-alkenyl, —O-alkynyl, —S-alkyl, —S-alkenyl, and —S-alkynyl;
R₆ is a radical selected from the group consisting of hydrogen, —CN, —COOH, —C(O)O-alkyl, —C(O)O-alkylenyl-R₄, —C(O)-alkyl, —C(O)-alkylenyl-R₄, —C(O)-halogen, —C(O)NH₂, —C(O)NH-alkyl, —C(O)NH-alkylenyl-R₄;
R₇ is a radical selected from the group consisting of O, NH, and S;
R₈ is N; and
R₉ is a radical selected from the group consisting of hydrogen, alkyl, alkenyl, alkynyl, cyclic rings, heterocyclic rings; and
R₂ and R₃ are each independently selected from the group consisting of hydrogen, alkyl, alkenyl, alkynyl, cyclic rings and heterocyclic rings,
or R₂ and R₃ together form a heterocyclic ring,
or R₃, together with Z₁ or Z₂ and the nitrogen to which R₃ is attached, forms a heterocyclic ring;
wherein when the compound is of the formula (IB) and X₂, Z₁, Z₂, Z₃, and Z₄ are all CH, X₃ is not CH;
and further wherein one or more of the hydrogen atoms are optionally replaced with a radiolabel.

2. A method according to claim 1, wherein the compound of formula (I) is radiolabeled with ¹⁸F, ⁷⁵Br, ⁷⁶Br, ¹²³I or ¹²⁴I.

3. A method according to claim 1, further comprising determining whether the structures are labeled by observing the brain tissue with a fluorescence microscope.

4. A method according to claim 1, wherein the compound according to claim 1 is radiolabeled.

5. A method according to claim 4, further comprising determining whether the structures are labeled by observing the brain tissue by a method capable of detecting and depicting the distribution of the radiolabeled compound within the body.

6. A method according to claim S, wherein the brain tissue is observed using positron emission tomography.

7. A method according to claim 1, wherein the compound has the formula IA.

8. A method according to claim 7, wherein Q is —NR₂R₃.

9. A method according to claim 7, wherein R₁ is —C(A₂)═C(CN)₂.

10. A method according to claim 9, wherein A₂ is alkyl.

11. A method according to claim 1, wherein the compound has the formula IB.

12. A method according to claim 11, wherein Q is —NR₂R₃.

13. A method according to claim 11, wherein R₃ and Z₁ together form a heterocyclic ring.

14. A method according to claim 11, wherein R₃ and Z₂ together form a heterocyclic ring.

15. A method according to claim 11, wherein the compound of formula IB is selected from the following compounds: 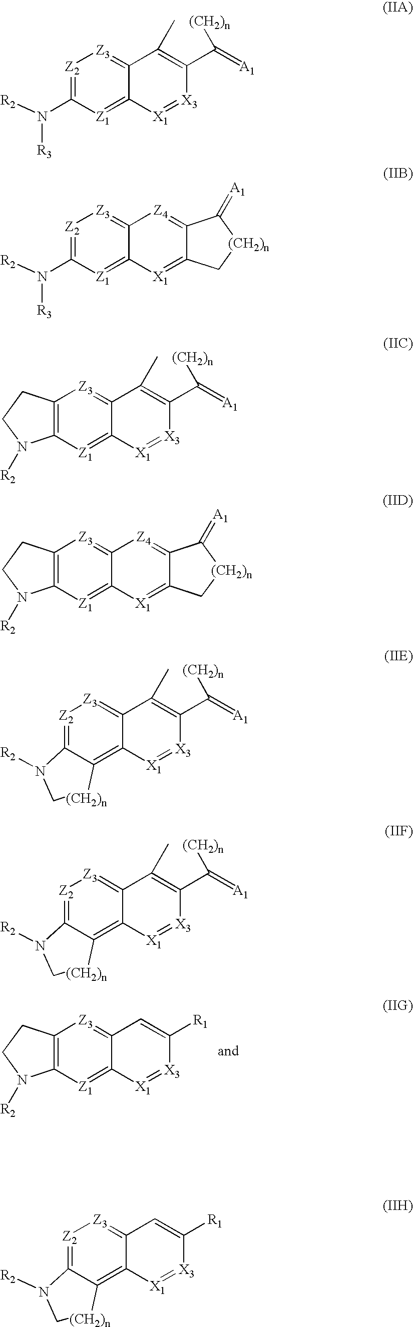
wherein n ranges from 1 to 3.

16. A method according to claim 11, wherein R₂ and R₃ together form a heterocyclic ring substituted with at least one radical selected from the group consisting of alkyl, alkenyl, alkynyl, alkoxy, OH, OTs, halogen, carbonyl, spiperone, spiperone ketal and spiperone-3-yl.

17. A method for determining the ability of a therapeutic agent to treat or prevent Alzheimer's disease, the method comprising:
(1) contacting, in vivo or in vitro, β-amyloid peptide with the therapeutic agent and a radiolabeled compound according to formula (IA) or (IB): 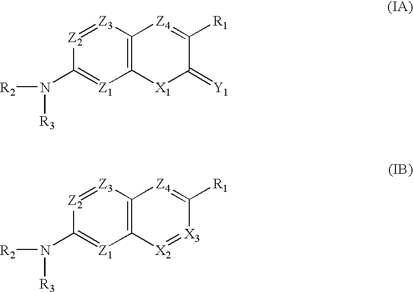
wherein:
X₁ is selected from the group consisting of O, S, —NR₄, —CH₂, —CH-alkyl, and —C(alkyl)₂;
X₂ is selected from the group consisting of N, —CH, and —C-alkyl;
X₃ is selected from the group consisting of O, S, —NR₄, and —C—Y₂;
Y₁ is selected from the group consisting of O, S, —NH, —CH₂, —CH-alkyl, and —C(alkyl)₂;
Y₂ is selected from the group consisting of H, OH, SH, —NH₂, —NH-alkyl, —N(alkyl)₂, —O-alkyl, and alkyl;
Z₁, Z₂, Z₃, and Z₄ are each independently selected from the group consisting of N, —CH, and —C-alkyl;
R₁ is selected from the group consisting of —CN, —C(=A₁)-A₂, 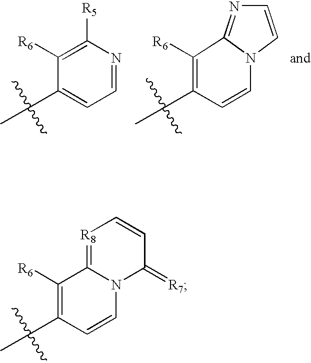
wherein:
A₁ is selected from the group consisting of O, S, —NR₄, —C(CN)₂, —C(CN)COOR₄, and —C(COOR₄)₂, and
A₂ is selected from the group consisting of OH, —O-alkyl, —NH₂, —NH—R₄, —N(R₄)₂, halogen, alkyl, cyclic rings, heterocyclic rings, —O-alkylenyl-R₄, —NH-alkylenyl-R₄, -alkylenyl-R₄, -alkylenyl-NHR₄, -alkylenyl-NH₂, and -alkylenyl-N(R₄)₂,
or A₁ and A₂ together form a cyclic ring or heterocyclic ring,
or R₁ together with X₃ or Z₄ forms a cyclic ring or heterocyclic ring substituted with an A, substitutent at a carbon other than R₁, X₃ or Z₄;
R₄ is a radical selected from the group consisting of alkyl, substituted alkyl, cyclic rings, substituted aryl, —C(O)O-alkyl, OH, OTs, and halogen;
R₅ is a radical selected from the group consisting of —NH₂, —OH, —OTs, —SH, halogen, —NH-alkyl, —NHR₄, —NH-alkylenyl-R₄, alkyl-(O-alkyl)₂, —O-alkyl, —O-alkylenyl-R₄, —S-alkyl, and —S-alkylenyl-R₄;
R₆ is a radical selected from the group consisting of —CN, —COOH, —C(O)O-alkyl, —C(O)O-alkylenyl-R₄, —C(O)-alkyl, —C(O)-alkylenyl-R₄, —C(O)-halogen, —C(O)NH₂, —C(O)NH-alkyl, —C(O)NH-alkylenyl-R₄;
R₇ is a radical selected from the group consisting of O, NH, and S; and
R₈ is N; and
R₂ and R₃ are each independently selected from the group consisting of alkyl and alkylenyl-R₁₀, wherein R₁₀ is selected from the group consisting of —OH, —OTs, halogen, spiperone, spiperone ketal, and spiperone-3-yl,
or R₂ and R₃ together form a heterocyclic ring, optionally substituted with at least one radical selected from the group consisting of alkyl, alkoxy, OH, OTs, halogen, alkylenyl-R₅, carbonyl, spiperone, spiperone ketal and spiperone-3-yl,
or R₃, together with Z₁ or Z₂ and the nitrogen to which R₃ is attached, forms a heterocyclic ring or a substituted heterocyclic ring;
wherein one or more of the hydrogen, halogen or carbon atoms is replaced with a radiolabel,
whereby at least some of the radiolabeled compound binds to the β-amyloid peptide; and
(2) determining the amount of radiolabeled compound that did not bind to the β-amyloid peptide to thereby determine the extent to which the therapeutic agent did bind to the β-amyloid peptide.

18. The method according to claim 17, wherein the therapeutic agent is first contacted with the β-amyloid peptide, and thereafter the radiolabeled compound is contacted with the β-amyloid peptide.

19. The method according to claim 17, comprising:
first contacting the radiolabeled compound with the β-amyloid peptide and determining the extent to which the radiolabeled compound binds to the β-amyloid peptide;
thereafter administering the therapeutic agent over a period of time to permit the therapeutic agent to contact the β-amyloid peptide; and
again contacting the radiolabeled compound with the β-amyloid peptide and determining the extent to which the radiolabeled compound binds to the β-amyloid peptide.

20. A method for determining the ability of a therapeutic agent to bind to β-amyloid peptide, the method comprising:
(1) contacting, in vivo or in vitro, β-amyloid peptide with the therapeutic agent and a radiolabeled compound according to formula (IA) or (IB): 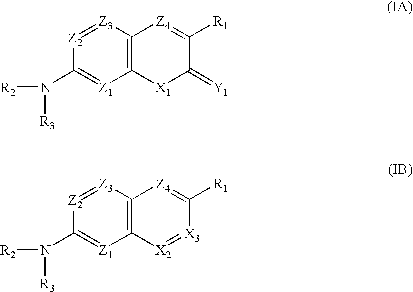
wherein:
X₁ is selected from the group consisting of O, S, —NR₄, —CH₂, —CH-alkyl, and —C(alkyl)₂;
X₂ is selected from the group consisting of N, —CH, and —C-alkyl;
X₃ is selected from the group consisting of O, S, —NR₄, and —C—Y₂;
Y₁ is selected from the group consisting of O, S, —NH, —CH₂, —CH-alkyl, and —C(alkyl)₂;
Y₂ is selected from the group consisting of H, OH, SH, —NH₂, —NH-alkyl, —N(alkyl)₂, —O-alkyl, and alkyl;
Z₁, Z₂, Z₃, and Z₄ are each independently selected from the group consisting of N, —CH, and —C-alkyl;
R₁ is selected from the group consisting of —CN, —C(=A₁)-A₂, 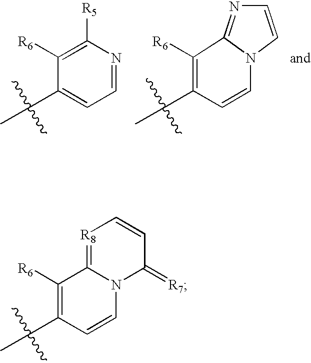
wherein:
A₁ is selected from the group consisting of O, S, —NR₄, —C(CN)₂, —C(CN)COOR₄, and —C(COOR₄)₂, and
A₂ is selected from the group consisting of OH, —O-alkyl, —NH₂, —NH—R₄, —N(R₄)₂, halogen, alkyl, cyclic rings, heterocyclic rings, —O-alkylenyl-R₄, —NH-alkylenyl-R₄, -alkylenyl-R₄, -alkylenyl-NHR₄, -alkylenyl-NH₂, and -alkylenyl-N(R₄)₂,
or A₁ and A₂ together form a cyclic ring or heterocyclic ring,
or R₁ together with X₃ or Z₄ forms a cyclic ring or heterocyclic ring substituted with an A₁ substitutent at a carbon other than R₁, X₃ or Z₄;
R₄ is a radical selected from the group consisting of alkyl, substituted alkyl, cyclic rings, substituted aryl, —C(O)O-alkyl, OH, OTs, and halogen;
R₅ is a radical selected from the group consisting of —NH₂, —OH, —OTs, —SH, halogen, —NH-alkyl, —NHR₄, —NH-alkylenyl-R₄, alkyl-(O-alkyl)₂, —O-alkyl, —O-alkylenyl-R₄, —S-alkyl, and —S-alkylenyl-R₄;
R₆ is a radical selected from the group consisting of —CN, —COOH, —C(O)O-alkyl, —C(O)O-alkylenyl-R₄, —C(O)-alkyl, —C(O)-alkylenyl-R₄, —C(O)-halogen, —C(O)NH₂, —C(O)NH-alkyl, —C(O)NH-alkylenyl-R₄;
R₇ is a radical selected from the group consisting of O, NH, and S; and
R₈ is N; and
R₂ and R₃ are each independently selected from the group consisting of alkyl and alkylenyl-R₁₀, wherein R₁₀ is selected from the group consisting of —OH, —OTs, halogen, spiperone; spiperone ketal, and spiperone-3-yl,
or R₂ and R₃ together form a heterocyclic ring, optionally substituted with at least one radical selected from the group consisting of alkyl, alkoxy, OH, OTs, halogen, alkylenyl-R₅, carbonyl, spiperone, spiperone ketal and spiperone-3-yl,
or R₃, together with Z₁ or Z₂ and the nitrogen to which R₃ is attached, forms a heterocyclic ring or a substituted heterocyclic ring;
wherein one or more of the hydrogen, halogen or carbon atoms is replaced with a radiolabel,
whereby at least some of the radiolabeled compound binds to the β-amyloid peptide; and
(2) determining the amount of radiolabeled compound that did not bind to the β-amyloid peptide to thereby determine the extent to which the therapeutic agent did bind to the β-amyloid peptide.

21. A method for treating or preventing Alzheimer's disease in a patient, the method comprising administering to the patient a therapeutically effective amount of an agent according to formula (IA) or (IB): 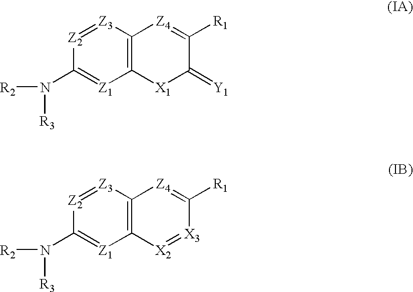
wherein:
X₁ is selected from the group consisting of O, S, —NR₄, —CH₂, —CH-alkyl, and —C(alkyl)₂;
X₂ is selected from the group consisting of N, —CH₁, and —C-alkyl;
X₃ is selected from the group consisting of O, S, —NR₄, and —C—Y₂;
Y₁ is selected from the group consisting of O, S, —NH, —CH₂, —CH-alkyl, and —C(alkyl)₂;
Y₂ is selected from the group consisting of H, OH, SH, —NH₂, —NH-alkyl, —N(alkyl)₂, —O-alkyl, and alkyl;
Z₁, Z₂, Z₃, and Z₄ are each independently selected from the group consisting of N, —CH, and —C-alkyl;
R₁ is selected from the group consisting of —CN, —C(=A₁)-A₂, 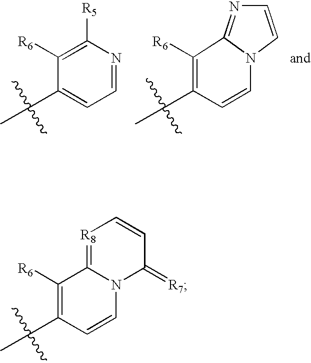
wherein:
A₁ is selected from the group consisting of O, S, —NR₄, —C(CN)₂, —C(CN)COOR₄, and —C(COOR₄)₂, and
A₂ is selected from the group consisting of OH, —O-alkyl, —NH₂, —NH—R₄, —N(R₄)₂, halogen, alkyl, cyclic rings, heterocyclic rings, —O-alkylenyl-R₄, —NH-alkylenyl-R₄, -alkylenyl-R₄, -alkylenyl-NHR₄, -alkylenyl-NH₂, and -alkylenyl-N(R₄)₂,
or A₁ and A₂ together form a cyclic ring or heterocyclic ring,
or R₁ together with X₃ or Z₄ forms a cyclic ring or heterocyclic ring substituted with an A₁ substitutent at a carbon other than R₁, X₃ or Z₄;
R₄ is a radical selected from the group consisting of alkyl, substituted alkyl, cyclic rings, substituted aryl, —C(O)O-alkyl, OH, OTs, and halogen;
R₅ is a radical selected from the group consisting of —NH₂, —OH, —OTs, —SH, halogen, —NH-alkyl, —NH₄, —NH-alkylenyl-R₄, alkyl-(O-alkyl)₂, —O-alkyl, —O-alkylenyl-R₄, —S-alkyl, and —S-alkylenyl-R₄;
R₆ is a radical selected from the group consisting of —CN, —COOH, —C(O)O-alkyl, —C(O)O-alkylenyl-R₄, —C(O)-alkyl, —C(O)-alkylenyl-R₄, —C(O)-halogen, —C(O)NH₂, —C(O)NH-alkyl, —C(O)NH-alkylenyl-R₄;
R₇ is a radical selected from the group consisting of O, NH, and S; and
R₈ is N; and
R₂ and R₃ are each independently selected from the group consisting of alkyl and alkylenyl-R₁₀, wherein R₁₀ is selected from the group consisting of —OH, —OTs, halogen, spiperone, spiperone ketal, and spiperone-3-yl,
or R₂ and R₃ together form a heterocyclic ring, optionally substituted with at least one radical selected from the group consisting of alkyl, alkoxy, OH, OTs, halogen, alkylenyl-R₅, carbonyl, spiperone, spiperone ketal and spiperone-3-yl,
or R₃, together with Z₁ or Z₂ and the nitrogen to which R₃ is attached, forms a heterocyclic ring or a substituted heterocyclic ring.

22. A composition comprising a compound of formula (IA) or (IB): 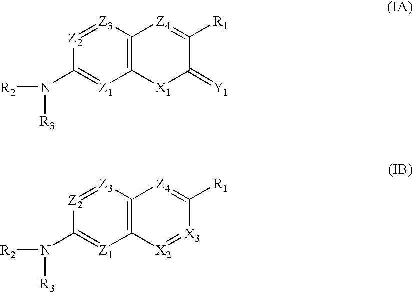
wherein:
X₁ is selected from the group consisting of O, S, —NR₄, —CH₂, —CH-alkyl, and —C(alkyl)₂;
X₂ is selected from the group consisting of N, —CH, and —C-alkyl;
X₃ is selected from the group consisting of O, S, —NR₄, and —C—Y₂;
Y₁ is selected from the group consisting of O, S, —NH, —CH₂, —CH-alkyl, and —C(alkyl)₂;
Y₂ is selected from the group consisting of H, OH, SH, —NH₂, —NH-alkyl, —N(alkyl)₂, —O-alkyl, and alkyl;
Z₁, Z₂, Z₃, and Z₄ are each independently selected from the group consisting of N, —CH, and —C-alkyl;
R₁ is selected from the group consisting of —CN, —C(=A₁)-A₂, 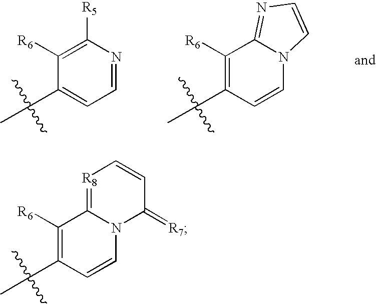
wherein:
A₁ is selected from the group consisting of O, S, —NR₄, —C(CN)₂, —C(CN)COOR₄, and —C(COOR₄)₂, and
A₂ is selected from the group consisting of OH, —O-alkyl, —NH₂, —NH—R₄, —N(R₄)₂, halogen, alkyl, cyclic rings, heterocyclic rings, —O-alkylenyl-R₄, —NH-alkylenyl-R₄, -alkylenyl-R₄, -alkylenyl-NHR₄, -alkylenyl-NH₂, and -alkylenyl-N(R₄)₂,
or A₁ and A₂ together form a cyclic ring or heterocyclic ring,
or R₁ together with X₃ or Z₄ forms a cycloalkyl or heterocyclic ring substituted with an A₁ substituted at a carbon other than R₁, X₃ or Z₄;
R₄ is a radical selected from the group consisting of alkyl, substituted alkyl, aryl, substituted aryl, —C(O)O-alkyl, OH, OTs, and halogen;
R₅ is a radical selected from the group consisting of —NH₂, —OH, —OTs, —SH, halogen, —NH-alkyl, —NHR₄, —NH-alkylenyl-R₄, alkyl-(O-alkyl)₂, —O-alkyl, —O-alkylenyl-R₄, —S-alkyl, and —S-alkylenyl-R₄;
R₆ is a radical selected from the group consisting of —CN, —COOH, —C(O)O-alkyl, —C(O)O-alkylenyl-R₄, —C(O)-alkyl, —C(O)-alkylenyl-R₄, —C(O)-halogen, —C(O)NH₂, —C(O)NH-alkyl, —C(O)NH-alkylenyl-R₄;
R₇ is a radical selected from the group consisting of O, NH, and S; and
R₈ is N; and
R₂ and R₃ are each independently selected from the group consisting of alkyl and alkylenyl-R₁₀, wherein R₁₀ is selected from the group consisting of —OH, —OTs, halogen, spiperone, spiperone ketal, and spiperone-3-yl,
or R₂ and R₃ together form a heterocyclic ring, optionally substituted with at least one radical selected from the group consisting of alkyl, alkoxy, OH, OTs, halogen, alkylenyl-R₅, carbonyl, spiperone, spiperone ketal and spiperone-3-yl,
or R₃, together with Z₁ or 72 and the nitrogen to which R₃ is attached, forms
a heterocyclic ring or a substituted heterocyclic ring;
and wherein:
when the compound is of the formula (IA), X₁ is O, and Y₁ is O, A₁ is not O;
when the compound is of the formula (IB) and X₂, Z₁, Z₂, Z₃, and Z₄ are all hydrogen, X₃ is not CH;
when the compound has formula IB and R₁ is —C(═C(CN)₂)-A₂,(1) at least one of Z₁, Z₂, Z₃, and Z₄ is N or —C-alkyl, (2) X₂ is N, or (3) Y₂ is selected from the group consisting of OH, SH, —NH₂, —NH-alkyl, —N(alkyl)₂, —O-alkyl, and alkyl;
and further wherein one or more of the hydrogen, halogen or carbon atoms are optionally replaced with a radiolabel.

23. A composition according to claim 22, wherein the compound of formula (I) is radiolabeled with ¹⁸F or ¹²³I.
